SEQUENCE LISTING (2 sequences)

SEQ ID NO: 1 - protein, 141 aa, Homo sapiens
MGFQKFSPFLALSILVLLQAGSLHAAPFRSALESSPADPATLSEDEARLLLAALVQDYVQMKASELEQEQEREGSSLDSPRSKRCGNLSTCMLGTYTQDFNKFHTFPQTAIGVGAPGKKRDMSSDLERDHRPHVSMPQNAN

SEQ ID NO: 2 - protein, 2768 aa, Homo sapiens
MALVLEIFTLLASICWVSANIFEYQVDAQPLRPCELQRETAFLKQADYVPQCAEDGSFQTVQCQNDGRSCWCVGANGSEVLGSRQPGRPVACLSFCQLQKQQILLSGYINSTDTSYLPQCQDSGDYAPVQCDVQQVQCWCVDAEGMEVYGTRQLGRPKRCPRSCEIRNRRLLHGVGDKSPPQCSAEGEFMPVQCKFVNTTDMMIFDLVHSYNRFPDAFVTFSSFQRRFPEVSGYCHCADSQGRELAETGLELLLDEIYDTIFAGLDLPSTFTETTLYRILQRRFLAVQSVISGRFRCPTKCEVERFTATSFGHPYVPSCRRNGDYQAVQCQTEGPCWCVDAQGKEMHGTRQQGEPPSCAEGQSCASERQQALSRLYFGTSGYFSQHDLFSSPEKRWASPRVARFATSCPPTIKELFVDSGLLRPMVEGQSQQFSVSENLLKEAIRAIFPSRGLARLALQFTTNPKRLQQNLFGGKFLVNVGQFNLSGALGTRGTFNFSQFFQQLGLASFLNGGRQEDLAKPLSVGLDSNSSTGTPEAAKKDGTMNKPTVGSFGFEINLQENQNALKFLASLLELPEFLLFLQHAISVPEDVARDLGDVMETVLSSQTCEQTPERLFVPSCTTEGSYEDVQCFSGECWCVNSWGKELPGSRVRGGQPRCPTDCEKQRARMQSLMGSQPAGSTLFVPACTSEGHFLPVQCFNSECYCVDAEGQAIPGTRSAIGKPKKCPTPCQLQSEQAFLRTVQALLSNSSMLPTLSDTYIPQCSTDGQWRQVQCNGPPEQVFELYQRWEAQNKGQDLTPAKLLVKIMSYREAASGNFSLFIQSLYEAGQQDVFPVLSQYPSLQDVPLAALEGKRPQPRENILLEPYLFWQILNGQLSQYPGSYSDFSTPLAHFDLRNCWCVDEAGQELEGMRSEPSKLPTCPGSCEEAKLRVLQFIRETEEIVSASNSSRFPLGESFLVAKGIRLRNEDLGLPPLFPPREAFAEQFLRGSDYAIRLAAQSTLSFYQRRRFSPDDSAGASALLRSGPYMPQCDAFGSWEPVQCHAGTGHCWCVDEKGGFIPGSLTARSLQIPQCPTTCEKSRTSGLLSSWKQARSQENPSPKDLFVPACLETGEYARLQASGAGTWCVDPASGEELRPGSSSSAQCPSLCNVLKSGVLSRRVSPGYVPACRAEDGGFSPVQCDQAQGSCWCVMDSGEEVPGTRVTGGQPACESPRCPLPFNASEVVGGTILCETISGPTGSAMQQCQLLCRQGSWSVFPPGPLICSLESGRWESQLPQPRACQRPQLWQTIQTQGHFQLQLPPGKMCSADYADLLQTFQVFILDELTARGFCQIQVKTFGTLVSIPVCNNSSVQVGCLTRERLGVNVTWKSRLEDIPVASLPDLHDIERALVGKDLLGRFTDLIQSGSFQLHLDSKTFPAETIRFLQGDHFGTSPRTWFGCSEGFYQVLTSEASQDGLGCVKCPEGSYSQDEECIPCPVGFYQEQAGSLACVPCPVGRTTISAGAFSQTHCVTDCQRNEAGLQCDQNGQYRASQKDRGSGKAFCVDGEGRRLPWWETEAPLEDSQCLMMQKFEKVPESKVIFDANAPVAVRSKVPDSEFPVMQCLTDCTEDEACSFFTVSTTEPEISCDFYAWTSDNVACMTSDQKRDALGNSKATSFGSLRCQVKVRSHGQDSPAVYLKKGQGSTTTLQKRFEPTGFQNMLSGLYNPIVFSASGANLTDAHLFCLLACDRDLCCDGFVLTQVQGGAIICGLLSSPSVLLCNVKDWMDPSEAWANATCPGVTYDQESHQVILRLGDQEFIKSLTPLEGTQDTFTNFQQVYLWKDSDMGSRPESMGCRKDTVPRPASPTEAGLTTELFSPVDLNQVIVNGNQSLSSQKHWLFKHLFSAQQANLWCLSRCVQEHSFCQLAEITESASLYFTCTLYPEAQVCDDIMESNAQGCRLILPQMPKALFRKKVILEDKVKNFYTRLPFQKLMGISIRNKVPMSEKSISNGFFECERRCDADPCCTGFGFLNVSQLKGGEVTCLTLNSLGIQMCSEENGGAWRILDCGSPDIEVHTYPFGWYQKPIAQNNAPSFCPLVVLPSLTEKVSLDSWQSLALSSVVVDPSIRHFDVAHVSTAATSNFSAVRDLCLSECSQHEACLITTLQTQPGAVRCMFYADTQSCTHSLQGQNCRLLLREEATHIYRKPGISLLSYEASVPSVPISTHGRLLGRSQAIQVGTSWKQVDQFLGVPYAAPPLAERRFQAPEPLNWTGSWDASKPRASCWQPGTRTSTSPGVSEDCLYLNVFIPQNVAPNASVLVFFHNTMDREESEGWPAIDGSFLAAVGNLIVVTASYRVGVFGFLSSGSGEVSGNWGLLDQVAALTWVQTHIRGFGGDPRRVSLAADRGGADVASIHLLTARATNSQLFRRAVLMGGSALSPAAVISHERAQQQAIALAKEVSCPMSSSQEVVSCLRQKPANVLNDAQTKLLAVSGPFHYWGPVIDGHFLREPPARALKRSLWVEVDLLIGSSQDDGLINRAKAVKQFEESRGRTSSKTAFYQALQNSLGGEDSDARVEAAATWYYSLEHSTDDYASFSRALENATRDYFIICPIIDMASAWAKRARGNVFMYHAPENYGHGSLELLADVQFALGLPFYPAYEGQFSLEEKSLSLKIMQYFSHFIRSGNPNYPYEFSRKVPTFATPWPDFVPRAGGENYKEFSELLPNRQGLKKADCSFWSKYISSLKTSADGAKGGQSAESEEEELTAGSGLREDLLSLQEPGSKTYSK

(19) United States
(12) Patent Application Publication
(10) Pub. No.: US 2024/0319180 A1
(43) Pub. Date: Sep. 26, 2024

(54) DEVICES AND METHODS FOR DIAGNOSISING THYROID MEDICAL CONDITIONS

(71) Applicants: B. G. NEGEV TECHNOLOGIES AND APPLICATIONS LTD., AT BEN-GURION UNIVERSITY, Beer Sheva (IL); MOR RESEARCH APPLICATIONS LTD., Ramat Gan (IL)

(72) Inventors: Robert MARKS, Omer (IL); Uri YOEL, Beer-Sheva (IL); Tim AXELROD, Haniel (IL); Sagi ANGEL, Rishon Lezion (IL); Ariel KUSHMARO, Beer Yaakov (IL)

(21) Appl. No.: 18/272,591

(22) PCT Filed: Jan. 17, 2022
(86) PCT No.: PCT/IL2022/050067

§ 371 Date: Jul. 16, 2023

Related U.S. Application Data
(60) Provisional application No. 63/138,481, filed on Jan. 17, 2021.

(51) Int. Cl. G01N 33/543; A61K 45/06; B01L 3/00; G01N 33/574; G01N 33/68; G01N 33/78

(52) U.S. Cl. CPC G01N 33/54388; A61K 45/06; B01L 3/5023; G01N 33/57407; G01N 33/6893; G01N 33/78; B01L 2300/0825; G01N 2333/585; G01N 2333/635

(57) ABSTRACT

Disclosed is a device including at least 4 sections with a unique layout which includes a surface functionalized with an agent having specific binding affinity to a target molecule, and which allows flow. Disclosed are also a kit and a method for determining and quantifying the presence of a biomarker of a thyroid medical condition in a sample.

### CROSS-REFERENCE TO RELATED APPLICATIONS

This application claims the benefit of priority to U.S. Provisional Patent Application No. 63/138,481, titled “DEVICES AND METHODS FOR DIAGNOSING THYROID MEDICAL CONDITIONS”, filed Jan. 17, 2021, the contents of which are incorporated herein by reference in their entirety.

### FIELD OF THE INVENTION

The present invention is in the field of thyroid pathology diagnosis, and is directed, in some embodiments, to devices and methods of using same for such diagnosis.

### BACKGROUND

Differentiated thyroid cancer (DTC), which includes papillary thyroid carcinoma (PTC) and follicular thyroid carcinoma (FTC), comprises the vast majority (>90%) of all thyroid cancers. Over the last 3 decades an increase in the incidence of DTC has been observed, mostly attributed to an increase in the incidence of PTC. It is expected that by 2030, DTC will become the fourth most common cancer in the general population and the second most common among women. Although PTCs have a favorable prognosis, persistent or recurrent disease, mainly involving the cervical lymph node compartments, is a common clinical problem, and may lead to local morbidity. As a result, disease-free survival has replaced overall survival as a main outcome of interest.

Serum and tissue thyroglobulin levels serve as a biomarker for DTC, since it is synthesized exclusively by thyroid follicular cells. Fine needle aspiration washout fluid for thyroglobulin measurement (FNAWF-Tg), is recommended together with fine needle aspiration biopsy (FNAB) for cytology in the diagnostic algorithm of cervical lymph nodes suspected as metastatic DTC. This recommendation is applied both before and after the primary treatment (thyroidectomy with or without radioactive iodine treatment) for DTC. Collectively, FNAWF-Tg demonstrated a sensitivity and specificity of over 90% for the detection of metastases to cervical lymph nodes. In some cases (e.g., cystic lymph nodes), FNAWF-Tg detection rate exceeded that of cytology, thus considered superior and complementary to FNAB for cytology. As of today, FNAWF-Tg is measured using the same assay that is used for measuring serum Tg, and therefore, the results are not available during the FNA procedure. From a technical point of view, FNAWF-Tg is performed following the dilution of the FNAWF-Tg needle content with 1 mL of 0.9% saline (the most accepted method); a Tg concentration above 10 ng/mL in such a sample is considered diagnostic for lymph node metastasis, while a concentration below 1 ng/ml makes the diagnosis of malignancy unlikely. Concentrations ranging from 1-10 ng/mL should be interpreted as moderately suspicious.

Despite the high validity of FNAWF-Tg when performed in the recommended clinical settings, there is a lack of standardization in different studies concerning patient selection (e.g., before or following thyroidectomy), biopsy technique (e.g., the amount of saline used to dilute the washout material in the syringe), and analytical method problems (e.g., to what degree, if any, anti-Tg-antibodies may interfere with the results). Some of these technical and analytical challenges may be resolved if Tg level measurement will be performed immediately following the procedure.

There is still a great need for a point of care (POC) assay for a biomarker of a thyroid medical condition, e.g., Tg (e.g., DTC), calcitonin (e.g., MTC), and a parathyroid hormone (e.g., intrathyroidal adenoma), which provides sensitive and accurate results within a few minutes.

### SUMMARY

According to a first aspect, there is provided a device for rapid diagnosis of a thyroid medical condition comprising at least 4 sections comprising: section 1, section 2, section 3, and section 4, sequentially and linearly coupled to each other, wherein: (a) section 1 comprises a sample collecting surface; (b) section 2 comprises at least one probing molecule having specific binding affinity to at least one biomarker of a thyroid medical condition being selected from the group consisting of: calcitonin, a parathyroid hormone, fragments thereof, and any combination thereof, wherein the at least one probing molecule is linked to a reporter molecule capable of generating a trigger; (c) section 3 comprises a surface functionalized with the at least one biomarker of a thyroid medical condition; and (d) section 4 comprises a surface comprising a substrate molecule capable of producing a signal upon contacting with the at least one reporter molecule linked to the probing molecule, wherein sections 1 to 4 are in liquid communication allowing flow of liquid sequentially from sections 1 to 4.

According to another aspect, there is provided a device for rapid diagnosis of a thyroid medical condition comprising at least 4 sections comprising: section 1, section 2, section 3, and section 4, sequentially and linearly coupled to each other, wherein: (a) section 1 comprises a sample collecting surface; (b) section 2 comprises at least one probing molecule having specific binding affinity to at least one biomarker of a thyroid medical condition being selected from the group consisting of: thyroglobulin, fragments thereof, and any combination thereof, wherein the at least one probing molecule is linked to a reporter molecule capable of generating a trigger, and wherein the at least one probing molecule comprises an antibody selected from the group consisting of: 138596-AF, and SC-366977; (c) section 3 comprises a surface functionalized with thyroglobulin; and (d) section 4 comprises a surface comprising a substrate molecule capable of producing a signal upon contacting with the at least one reporter molecule linked to the probing molecule, wherein sections 1 to 4 are in liquid communication allowing flow of liquid sequentially from sections 1 to 4.

According to another aspect, there is provided a method for diagnosing metastatic differentiating thyroid carcinoma (DTC) in a subject, comprising the steps of: (a) providing a sample comprising an extra thyroidal tissue or a fragment thereof derived from the subject; and (b) loading the sample from step (a) to the device of the invention, and detecting a signal produced by the substrate molecule, wherein detection of the signal is indicative of a presence of a biomarker of DTC in the sample, and wherein the presence of the biomarker of DTC in the sample is indicative of a cancerous thyroidal cell being present in the sample, thereby diagnosing metastatic DTC in the subject.

According to another aspect, there is provided a method for diagnosing medullary thyroid carcinoma (MTC) in a subject, comprising the steps of: (a) providing a sample comprising a thyroidal tissue or a fragment thereof derived from the subject; and (b) loading the sample from step (a) to the device of the invention, and detecting a signal produced by the substrate molecule, wherein detection of the signal is indicative of a presence of a biomarker of MTC in the sample, and wherein the presence of the biomarker of MTC in the sample is indicative of a cancerous thyroidal parafollicular cell being present in the sample, thereby diagnosing metastatic MTC in the subject.

According to another aspect, there is provided a kit for diagnosing a thyroid medical condition comprising at least 4 sections, comprising: (a) a section 1, a section 2, a section 3, and a section 4; (b) at least one biomarker of a thyroid medical condition selected from the group consisting of: thyroglobulin, calcitonin, a para-thyroid hormone, fragments thereof, and any combination thereof; (c) at least one probing molecule linked to a reporter molecule and having specific binding affinity to the at least one biomarker or a fragment thereof, wherein the reporter molecule generates: chemically-, electrically-, or physically-detectable reaction; and (d) a substrate molecule reacting in the presence of the reporter molecule.

In some embodiments, sections 1 to 4 are arranged along a horizontal axis.

In some embodiments, the flow is a lateral flow.

In some embodiments, sections 1 to 4 are arranged along a vertical axis.

In some embodiments, the flow is a longitudinal flow.

In some embodiments, the thyroid medical condition comprises thyroid cancer, metastases thereof, or a combination thereof.

In some embodiments, the at least one biomarker is a biomarker of medullary thyroid carcinoma (MTC).

In some embodiments, the at least one biomarker of MTC is a peptide comprising an amino acid sequence set forth in SEQ ID NO: 1, or a fragment thereof.

In some embodiments, the at least one probing molecule comprises an antibody selected from the group consisting of: DCABH-5057, MBS2107026, MBS2042771, MBS6250357, and MBS6250358.

In some embodiments, the at least one biomarker is a biomarker of differentiated thyroid carcinoma (DTC).

In some embodiments, the at least one biomarker of DTC is a peptide comprising an amino acid sequence set forth in SEQ ID NO: 2, or a fragment thereof.

In some embodiments, the reporter molecule is selected from the group consisting of: an enzyme, a radioactive molecule, a luminescent compound, a fluorescent compound, a magnetic particle, an electro-chemiluminescent compound, a fluorescence transducing aptamer and an electrochemically active compound.

In some embodiments, the device further comprises a calibration area disposed between section 2 and section 3, wherein the calibration area comprises a surface in contact with the substrate molecule.

In some embodiments, the device further comprises a detection unit in operable communication with the device, and wherein the detection unit is configured to detect the signal.

In some embodiments, the detection unit comprises an element selected form the group consisting of: an active-pixel sensor (APS), an electrode, an excitation source with active-pixel sensor, and any combination thereof.

In some embodiments, rapid diagnosis of the thyroid medical condition is provided within 1 minute to 30 minutes.

In some embodiments, DTC comprises any one of: papillary thyroid carcinoma (PTC), follicular thyroid carcinoma (FTC), and a combination thereof.

In some embodiments, the sample is devoid of a thyroid tissue.

In some embodiments, the extra thyroidal tissue or fragment thereof is selected form the group consisting of: a lymph node, a lung metastasis, a liver metastasis, a bone metastasis, a central nerve system (CNS) metastasis, and any combination thereof.

In some embodiments, the lymph node is a cervical lymph node, a mediastinal lymph node, or an axillary lymph node.

In some embodiments, the lymph node is abnormally enlarged, abnormally structured, or both, compared to a control lymph node.

In some embodiments, detecting comprises qualitatively determining, quantitatively determining, or both.

In some embodiments, the method further comprises determining a progression stage of the metastatic DTC in the subject.

In some embodiments, the method further comprises a step of treating the subject diagnosed with metastatic DTC with an effective amount of anti-metastatic DTC therapy.

In some embodiments, the anti-metastatic DTC therapy comprises: surgically removing an enlarged cervical lymph node of the subject, surgically removing at least a portion of a thyroid of the subject, surgically removing a metastasis from a site selected from the group consisting of: lung, liver, bone, CNS, and any combination thereof, administering to the subject a therapeutically effective amount of a drug suitable for DTC therapy, subjecting the subject to a therapeutically effective amount of radiotherapy, or any combination thereof.

In some embodiments, the drug is selected from the group consisting of: Vandetanib, Cabozantinib-S-Malate, Dabrafenib Mesylate, Doxorubicin Hydrochloride, Lenvatinib Mesylate, Trametinib, Sorafenib Tosylate, and Selpercatinib.

In some embodiments, the radiotherapy comprises internal radiotherapy or external radiotherapy.

In some embodiments, the internal radiotherapy comprises radiolabeled iodine.

In some embodiments, MTC comprises metastatic MTC.

In some embodiments, the thyroidal tissue or fragment thereof comprises a lymph node.

In some embodiments, the method further comprises determining a progression stage of the MTC in the subject.

In some embodiments, the method further comprises a step of treating the subject diagnosed with MTC with an effective amount of anti MTC therapy.

In some embodiments, the treating comprises: surgically removing an enlarged cervical lymph node of the subject, surgically removing at least a portion of a thyroid of the subject, surgically removing a metastasis from a site selected from the group consisting of: lung, liver, bone, CNS, and any combination thereof, administering to the subject a therapeutically effective amount of a drug suitable for MTC therapy, subjecting the subject to a therapeutically effective amount of an external radiotherapy, or any combination thereof.

In some embodiments, the kit further comprises a calibration area.

In some embodiments, the kit further comprises instructions for depositing: (a) section 2 with the reporter molecule; (b) section 3 with the at least one biomarker of a thyroid condition; and (c) section 4 with the substrate molecule.

In some embodiments, the at least one biomarker comprises a peptide comprising an amino acid sequence set forth in SEQ ID NO: 1, SEQ ID NO:2, or a fragment thereof.

In some embodiments, the thyroid medical condition comprises thyroid cancer.

In some embodiments, the thyroid cancer comprises metastatic DTC, MTC, or both.

In some embodiments, the at least one probing molecule is selected from any one of: (a) 138596-AF or SC-366977; and (b) DCABH-5057, MBS2107026, MBS2042771, MBS6250357, or MBS6250358.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 includes a scheme of a non-limiting clinical scenario according to which a subject is evaluated for thyroid nodule accompanied with suspicious cervical lymph node.

FIG. 2 includes a scheme of a non-limiting clinical scenario according to which a subject is evaluated for recurrent or persistent DTC following thyroidectomy (e.g., subject presents a suspicious cervical lymphadenopathy).

FIG. 3 includes a scheme of a non-limiting clinical scenario according to which, during an operation as a part of a primary treatment for PTC or for recurrent disease, a suspicious lymph node is observed, wherein it is unclear whether this lymph node represents a metastasis originating from PTC or a reactive lymph node.

FIG. 4 includes a scheme of a non-limiting clinical scenario according to which a subject is evaluated for suspected intrathyroidal parathyroid adenoma.

FIG. 5 includes a scheme of a non-limiting clinical scenario according to which a subject is evaluated for intermediate to high suspicious thyroid nodule.

FIG. 6 is a perspective view simplified illustration of a capture flow device, according to some embodiments of the present invention;

FIGS. 7A-7B are perspective view simplified illustrations of how the capture flow device works during an assay measurement according to some embodiments of the present invention; with a sample comprising or devoid of a biomarker of a thyroid medical condition (7A) or (7B), respectively.

FIG. 8 is a perspective view simplified illustration of a device comprising a calibration area, according to some embodiments of the present invention;

FIGS. 9A-9B are perspective view simplified illustrations of how the device comprising a calibration area works during an assay measurement according to some embodiments of the present invention with a sample comprising or devoid of a biomarker of a thyroid medical condition (9A) or (9B), respectively.

FIG. 10 is a perspective view simplified illustration of a device comprising a section 5, according to some embodiments of the present invention.

FIGS. 11A-11B include vertical bar graphs showing thyroglobulin calibration curve using 2 different antibodies, SC-366977 (11A) and 138597-af-HRP (11B). *p<0.05; **p<0.01.

FIGS. 12A-12B include vertical bar graphs and images showing thyroglobulin calibration curve (12A), and images of non-limiting examples of the device of the invention comprising 4 different anti-thyroglobulin antibodies (12B).

FIGS. 13A-13B include a schematic non-limiting organization of a lateral flow device according to some embodiments of the present invention (13A), and a schematic non-limiting assay performed using the device of 13A in the presence (“positive”, upper panel) or absence (“negative”, lower panel) of an analyte (13B).

FIG. 14 is a perspective view simplified illustration of lateral flow device, according to some embodiments of the present invention, comprising a plurality of calibration areas, e.g., located at two opposing sides of section 3.

FIG. 15 includes a scheme of a study design on thyroglobulin Point of Care assay for rapid detection of metastatic differentiated thyroid carcinoma, as described in Example 8.

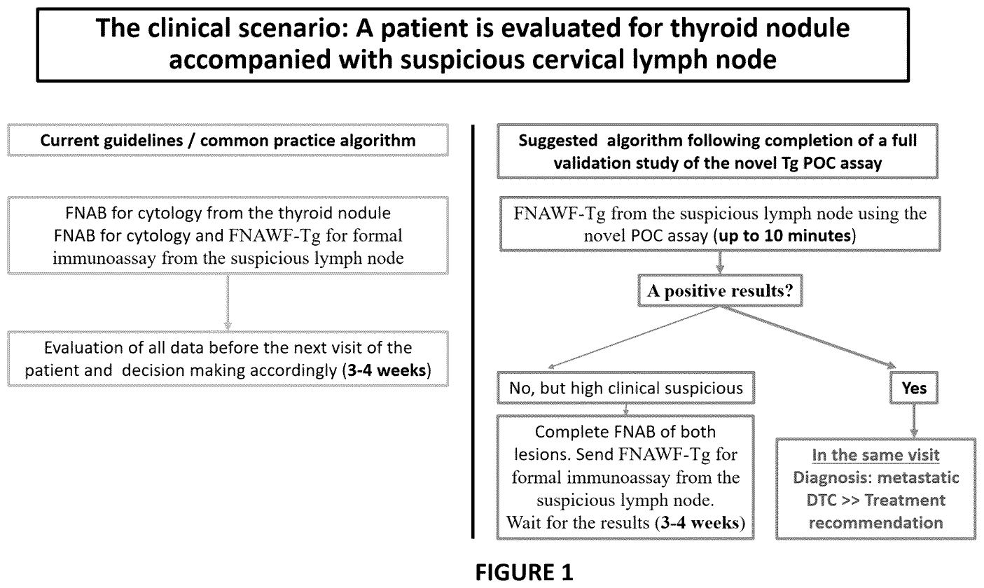

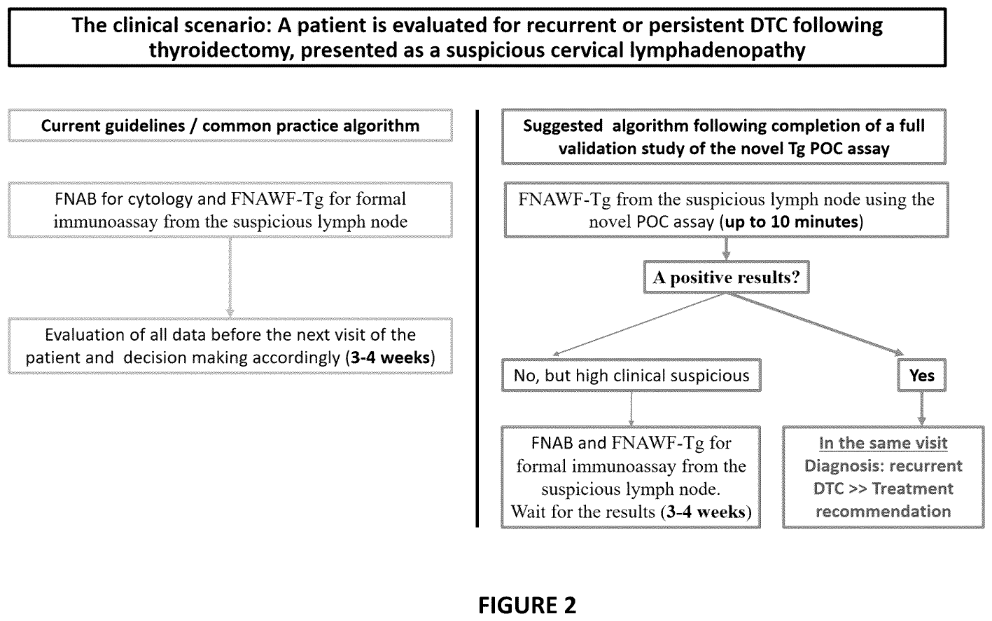

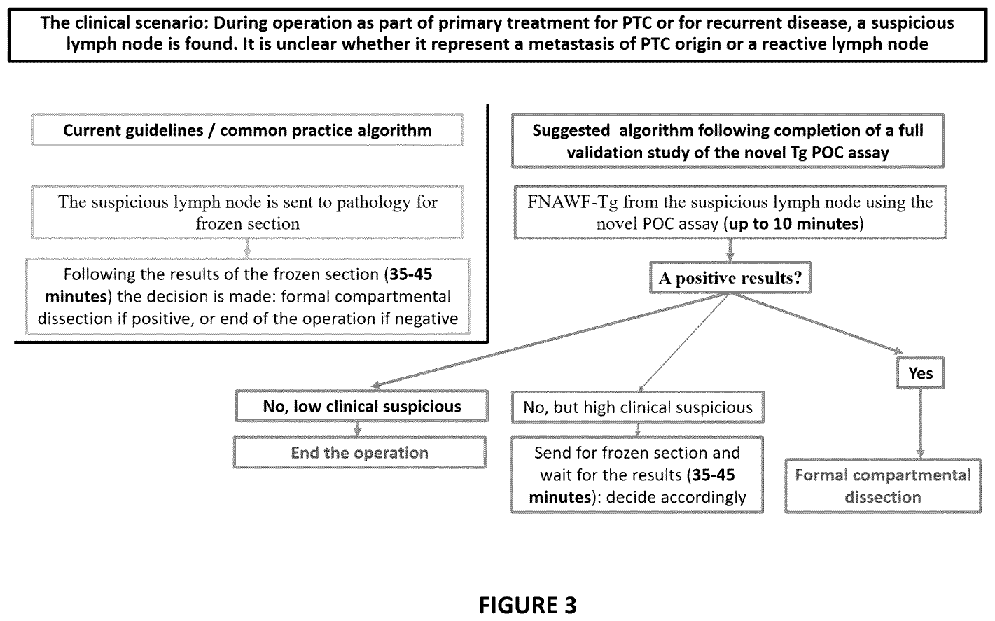

### DETAILED DESCRIPTION

The present invention, in some embodiments, relates to a flow device. In some embodiments, the device is a point of care testing device. In some embodiments, the present invention is directed to a rapid diagnosis of a thyroid medical condition, e.g., cancer. In some embodiments, the method further comprises treating a subject diagnosed with a thyroid medical condition, as disclosed hereinbelow.

### Devices for Rapid Diagnosis

According to some embodiments, there is provided a device for rapid diagnosis of a thyroid medical condition.

In some embodiments, the deice comprises at least 4 sections comprising: section 1, section 2, section 3, and section 4, sequentially and linearly coupled to each other.

In some embodiments, section 1 comprises a sample collecting surface.

In some embodiments, section 2 comprises at least one probing molecule having specific binding affinity to at least one biomarker of a thyroid medical condition.

In some embodiments, the at least one biomarker of a thyroid medical condition is selected from calcitonin, a parathyroid hormone (PTH), fragments thereof, and any combination thereof.

In some embodiments, the at least one probing molecule is linked to a reporter molecule capable of generating a trigger.

In some embodiments, section 3 comprises a surface functionalized with the at least one biomarker of a thyroid medical condition.

In some embodiments, section 4 comprises a surface comprising a substrate molecule capable of producing a signal upon contacting with the at least one reporter molecule linked to the probing molecule.

In some embodiments, sections 1 to 4 are in liquid communication allowing flow of liquid sequentially from sections 1 to 4.

According to some embodiments, there is provided a device for rapid diagnosis of a thyroid medical condition comprising at least 4 sections comprising: section 1, section 2, section 3, and section 4, sequentially and linearly coupled to each other as disclosed herein, wherein section 2 comprises at least one probing molecule having specific binding affinity to thyroglobulin, fragments thereof, or any combination thereof.

In some embodiments, the at least one probing molecule comprises an antibody selected from: 138596-AF or SC-366977. In some embodiments, the at least one probing molecule comprising an antibody as disclosed herein, has increased binding affinity and/or specificity to thyroglobulin, a fragment thereof, or a combination thereof.

In some embodiments, the at least one probing molecule comprises the antibody NB 110-8083.

In some embodiments, section 3 comprises a surface functionalized with thyroglobulin, fragments thereof, or any combination thereof.

In some embodiments, sections 1 to 4 are arranged along a horizontal axis.

In some embodiments, sections 1 to 4 are arranged along a vertical axis.

In some embodiments, the flow is a lateral flow.

In some embodiments, the flow is a longitudinal flow.

In some embodiments, the thyroid medical condition comprises thyroid cancer, metastases thereof, or a combination thereof.

In some embodiments, at least one biomarker is a biomarker of medullary thyroid carcinoma (MTC).

As used herein, the terms “Medullary thyroid carcinoma” or “MTC” refer to any thyroid carcinoma which originates from the parafollicular cells, which are characterized by the production of calcitonin.

In some embodiments, the at least one biomarker of MTC is calcitonin. In some embodiments, calcitonin comprises a mammalian calcitonin. In some embodiments, calcitonin comprises a human calcitonin.

In some embodiments, calcitonin comprises a peptide comprising an amino acid sequence set forth in: MGFQKFSPFLALSILVLLQAGSLHAAPFRSALESSPADPATLSEDEARLLLAALVQ DYVQMKASELEQEQEREGSSLDSPRSKRCGNLSTCMLGTYTQDFNKFHTFPQTAI GVGAPGKKRDMSSDLERDHRPHVSMPQNAN (SEQ ID NO: 1), or a fragment thereof.

In some embodiments, the at least one biomarker of MTC is a peptide comprising an amino acid sequence set forth in SEQ ID NO: 1, or a fragment thereof.

In some embodiments, the at least one probing molecule comprises an antibody selected from: DCABH-5057, MBS2107026, MBS2042771, MBS6250357, and MBS6250358.

In some embodiments, the at least one biomarker is a biomarker of differentiated thyroid carcinoma (DTC).

As used herein, the terms “Differentiated thyroid carcinoma” or “DTC” refer to any thyroid carcinoma which originates from differentiated follicular cells, which are characterized by the production of thyroglobulin.

In some embodiments, the at least one biomarker of DTC is thyroglobulin. In some embodiments, thyroglobulin comprises a mammalian thyroglobulin. In some embodiments, thyroglobulin comprises a human thyroglobulin. In some embodiments, thyroglobulin comprises a peptide comprising an amino acid sequence set forth in: MALVLEIFTLLASICWVSANIFEYQVDAQPLRPCELQRETAFLKQADYVPQCAED GSFQTVQCQNDGRSCWCVGANGSEVLGSRQPGRPVACLSFCQLQKQQILLSGYI NSTDTSYLPQCQDSGDYAPVQCDVQQVQCWCVDAEGMEVYGTRQLGRPKRCP RSCEIRNRRLLHGVGDKSPPQCSAEGEFMPVQCKFVNTTDMMIFDL VHSYNRFPD AFVTFSSFQRRFPEVSGYCHCADSQGRELAETGLELLLDEIYDTIFAGLDLPSTFTE TTLYRILQRRFLAVQSVISGRFRCPTKCEVERFTATSFGHPYVPSCRRNGDYQAVQ CQTEGPCWCVDAQGKEMHGTRQQGEPPSCAEGQSCASERQQALSRLYFGTSGYF SQHDLFSSPEKRWASPRVARFATSCPPTIKELFVDSGLLRPMVEGQSQQFSVSENL LKEAIRAIFPSRGLARLALQFTTNPKRLQQNLFGGKFLVNVGQFNLSGALGTRGTF NFSQFFQQLGLASFLNGGRQEDLAKPLSVGLDSNSSTGTPEAAKKDGTMNKPTV GSFGFEINLQENQNALKFLASLLELPEFLLFLQHAISVPEDVARDLGDVMETVLSS QTCEQTPERLFVPSCTTEGSYEDVQCFSGECWCVNSWGKELPGSRVRGGQPRCPT DCEKQRARMQSLMGSQPAGSTLFVPACTSEGHFLPVQCFNSECYCVDAEGQAIP GTRSAIGKPKKCPTPCQLQSEQAFLRTVQALLSNSSMLPTLSDTYIPQCSTDGQWR QVQCNGPPEQVFELYQRWEAQNKGQDLTPAKLLVKIMSYREAASGNFSLFIQSL YEAGQQDVFPVLSQYPSLQDVPLAALEGKRPQPRENILLEPYLFWQILNGQLSQY PGSYSDFSTPLAHFDLRNCWCVDEAGQELEGMRSEPSKLPTCPGSCEEAKLRVLQ FIRETEEIVSASNSSRFPLGESFLVAKGIRLRNEDLGLPPLFPPREAFAEQFLRGSDY AIRLAAQSTLSFYQRRRFSPDDSAGASALLRSGPYMPQCDAFGSWEPVQCHAGT GHCWCVDEKGGFIPGSLTARSLQIPQCPTTCEKSRTSGLLSSWKQARSQENPSPKD LFVPACLETGEYARLQASGAGTWCVDPASGEELRPGSSSSAQCPSLCNVLKSGVL SRRVSPGYVPACRAEDGGFSPVQCDQAQGSCWCVMDSGEEVPGTRVTGGQPAC ESPRCPLPFNASEVVGGTILCETISGPTGSAMQQCQLLCRQGSWSVFPPGPLICSLE SGRWESQLPQPRACQRPQLWQTIQTQGHFQLQLPPGKMCSADYADLLQTFQVFI LDELTARGFCQIQVKTFGTLVSIPVCNNSSVQVGCLTRERLGVNVTWKSRLEDIP VASLPDLHDIERAL VGKDLLGRFTDLIQSGSFQLHLDSKTFPAETIRFLQGDHFGTS PRTWFGCSEGFYQVLTSEASQDGLGCVKCPEGSYSQDEECIPCPVGFYQEQAGSL ACVPCPVGRTTISAGAFSQTHCVTDCQRNEAGLQCDQNGQYRASQKDRGSGKAF CVDGEGRRLPWWETEAPLEDSQCLMMQKFEKVPESKVIFDANAPVAVRSKVPDS EFPVMQCLTDCTEDEACSFFTVSTTEPEISCDFYAWTSDNVACMTSDQKRDALGN SKATSFGSLRCQVKVRSHGQDSPAVYLKKGQGSTTTLQKRFEPTGFQNMLSGLY NPIVFSASGANLTDAHLFCLLACDRDLCCDGFVLTQVQGGAIICGLLSSPSVLLCN VKDWMDPSEAWANATCPGVTYDQESHQVILRLGDQEFIKSLTPLEGTQDTFTNF QQVYLWKDSDMGSRPESMGCRKDTVPRPASPTEAGLTTELFSPVDLNQVIVNGN QSLSSQKHWLFKHLFSAQQANLWCLSRCVQEHSFCQLAEITESASLYFTCTLYPE AQVCDDIMESNAQGCRLILPQMPKALFRKKVILEDKVKNFYTRLPFQKLMGISIR NKVPMSEKSISNGFFECERRCDADPCCTGFGFLNVSQLKGGEVTCLTLNSLGIQM CSEENGGAWRILDCGSPDIEVHTYPFGWYQKPIAQNNAPSFCPLVVLPSLTEK VSL DSWQSLALSSVVVDPSIRHFDVAHVSTAATSNFSAVRDLCLSECSQHEACLITTLQ TQPGAVRCMFYADTQSCTHSLQGQNCRLLLREEATHIYRKPGISLLSYEASVPSVP ISTHGRLLGRSQAIQVGTSWKQVDQFLGVPYAAPPLAERRFQAPEPLNWTGSWD ASKPRASCWQPGTRTSTSPGVSEDCLYLNVFIPQNVAPNASVLVFFHNTMDREES EGWPAIDGSFLAAVGNLIVVTASYRVGVFGFLSSGSGEVSGNWGLLDQVAALTW VQTHIRGFGGDPRRVSLAADRGGADVASIHLLTARATNSQLFRRAVLMGGSALS PAAVISHERAQQQAIALAKEVSCPMSSSQEVVSCLRQKPANVLNDAQTKLLAVS GPFHYWGPVIDGHFLREPPARALKRSLWVEVDLLIGSSQDDGLINRAKAVKQFEE SRGRTSSKTAFYQALQNSLGGEDSDARVEAAATWYYSLEHSTDDYASFSRALEN ATRDYFIICPIIDMASAWAKRARGNVFMYHAPENYGHGSLELLADVQFALGLPFY PAYEGQFSLEEKSLSLKIMQYFSHFIRSGNPNYPYEFSRKVPTFATPWPDFVPRAG GENYKEFSELLPNRQGLKKADCSFWSKYISSLKTSADGAKGGQSAESEEEELTAG SGLREDLLSLQEPGSKTYSK (SEQ ID NO: 2), or a fragment thereof.

In some embodiments, the at least one biomarker of DTC is a peptide comprising an amino acid sequence set forth in SEQ ID NO: 2, or a fragment thereof.

As used herein, the term “analog” includes any peptide having an amino acid sequence substantially identical to one of the sequences specifically shown herein in which one or more residues have been conservatively substituted with a functionally similar residue and which displays the abilities as described herein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. Each possibility represents a separate embodiment of the present invention.

The term “analog” as used herein, refers to a peptide that is similar, but not identical, to any one of the herein disclosed peptides that still is capable of being bound by the probing molecule disclosed herein, e.g., an antibody. The analog may have deletions or mutations that result in an amino acids sequence that is different than the amino acid sequence of the herein disclosed peptides e.g., SEQ ID Nos.: 1-2. It should be understood that all analogs of the herein disclosed peptides would still be recognizable and specifically bound by the probing molecule, e.g., an antibody, as described herein. Further, an analog may be analogous to a fragment of the herein disclosed peptide, however, in such a case the fragment must comprise at least 10, at least 20, at least 30, at least 40, at least 50, at least 60, at least 70, at least 80, at least 90, or at least 100 consecutive amino acids of the herein disclosed peptide, or any value and range therebetween. Each possibility represents a separate embodiment of the invention.

In some embodiments, an analog to the peptide comprises an amino acid sequence with at least 10%, at least 15%, at least 20%, at least 25%, at least 30%, at least 40%, at least 50%, at least 60%, at least 65%, at least 70%, at least 75%, at least 80%, at least 85%, at least 90%, at least 95%, or at least 99% homology or identity to the amino acid sequence presented in SEQ ID NO: 1 or SEQ ID NO: 2, or any value and range therebetween. Each possibility represents a separate embodiment of the invention.

In some embodiments, the reporter molecule is selected from: an enzyme, a radioactive molecule, a luminescent compound, a fluorescent compound, a magnetic particle, an electro-chemiluminescent compound, a fluorescence transducing aptamer, or an electrochemically active compound.

In some embodiments, the device, further comprises a calibration area disposed between section 2 and section 3. In some embodiments, the device, further comprises a calibration area disposed between section 4 and section 5. In some embodiments, the device, further comprises a plurality of calibration areas. In some embodiments, any one of the calibration area or a plurality of calibration areas comprises a surface in contact with the substrate molecule.

In some embodiments, the device further comprises a detection unit in operable communication with the device. In some embodiments, the detection unit is configured to detect the signal.

In some embodiments, the detection unit comprises an element selected form: an active-pixel sensor (APS), an electrode, an excitation source with active-pixel sensor, or any combination thereof.

In some embodiments, rapid diagnosis of a thyroid medical condition is provided within 1 minute to 30 minutes, 1 minute to 5 minutes, 1 minute to 10 minutes, 1 minute to 15 minutes, 1 minute to 20 minutes, 1 minute to 25 minutes, 1 minute to 35 minutes, 1 minute to 40 minutes, 1 minute to 45 minutes, 5 minutes to 15 minutes, 5 minutes to 25 minutes, 10 minutes to 25 minutes, or 10 minutes to 35 minutes. Each possibility represents a separate embodiment of the invention.

In some embodiments, the term “coupled” as used herein comprises in contact with or in liquid communication. In some embodiments, section 1 comprises a sample collecting surface. In some embodiments, section 2 comprises a surface comprising a probing molecule having specific affinity to a biomarker of a thyroid medical condition linked to a reporter molecule, wherein the signal molecule generates a chemically and/or a physically detectable reaction. In some embodiments, any one of section 3 or 4 comprises a surface comprising a substrate molecule. In some embodiments, section 3 comprises a surface comprising a substrate molecule. In some embodiments, section 4 comprises a surface comprising a substrate molecule.

In some embodiments, the substrate molecule is positioned at least 1 mm, at least 2 mm, at least 3 mm, at least 4 mm, at least 5 mm, at least 6 mm, at least 7 mm, at least 8 mm, at least 9 mm, or at least 10 mm from the contacting point of section 3 with section 4, or any value and range therebetween. Each possibility represents a separate embodiment of the invention.

In some embodiments, the substrate molecule is positioned 1 mm to 15, 2 mm to 10 mm, 3 mm to 8 mm, or 3 mm to 6 mm from the contacting point of section 3 with section 4. Each possibility represents a separate embodiment of the invention.

In some embodiments, the substrate molecule is positioned about 4 mm from the contacting point of section 3 with section 4.

In some embodiments, the substrate molecule is positioned at least 1 mm, at least 2 mm, at least 3 mm, at least 4 mm, at least 5 mm, at least 6 mm, at least 7 mm, at least 8 mm, at least 9 mm, or at least 10 mm from the contacting point of section 4 with section 5, or any value and range therebetween. Each possibility represents a separate embodiment of the invention.

In some embodiments, the substrate molecule is positioned 1 mm to 15, 2 mm to 10 mm, 3 mm to 8 mm, or 3 mm to 6 mm from the contacting point of section 4 with section 5. Each possibility represents a separate embodiment of the invention.

In some embodiments, the substrate molecule is positioned about 4 mm from the contacting point of section 4 with section 5.

In some embodiments, section 2 comprises a surface in contact with a probing molecule having specific affinity to the biomarker of a thyroid medical condition, wherein the probing molecule is linked or bound to a reporter molecule, and wherein the reporter molecule generates a trigger. In some embodiments, section 4 comprises a surface in contact with a substrate molecule, wherein the substrate molecule generates a signal in response to the trigger generated by the reporter molecule.

In some embodiments, a device according to the present invention further comprises a section 5 coupled to and/or in liquid communication with section 4.

In some embodiments, section 1, section 2, section 3 section 4 and section 5 are partially overlapping. In some embodiments, section 1, section 2, section 3 section 4 and section 5 are partially overlapping, wherein overlapping comprises from 0.01% to 99%, from 0.01% to 95%, from 0.01% to 90%, from 1% to 90%, from 0.01% to 1%, from 1% to 80%, from 1% to 70%, from 1% to 60%, from 1% to 50%, from 1% to 40%, from 1% to 30%, from 1% to 20%, from 1% to 10%, from 1% to 5%, from 5% to 10%, from 10% to 20%, from 20% to 30%, from 30% to 40%, from 10% to 30%, from 10% to 40%, from 10% to 50%, from 10% to 60%, from 10% to 70%, of the total surface of the section.

In some embodiments, the terms “in liquid communication”, “in contact with” and “coupled” are used herein interchangeably.

As used herein, the term “point of care testing” refers to real time diagnostic testing that can be done in a rapid time frame so that the resulting test is performed faster than comparable tests that do not employ this system. It can be performed in a doctor's office, at a bedside, in a stat laboratory, emergency room, ambulances or at home and other such locales, particularly where rapid and accurate results are required. The patient can be present, but such presence is not required. Point of care includes, but is not limited to: an emergency room, an operating room, a hospital laboratory and/or any other clinical laboratory, a doctor's office, in the field, or in any situation in which a rapid and accurate result is desired.

According to some embodiments, there is provided a device which utilize specific binding members (probing element). The term “specific binding member” as used herein, refers to a member of a specific binding pair. That is, two different molecules where one of the molecules through chemical or physical means specifically binds to the second molecule. Therefore, in addition to antigen and antibody specific binding pairs of common immunoassays, other specific binding pairs can include biotin and avidin, carbohydrates and lectins, complementary nucleotide sequences, effector and receptor molecules, cofactors and enzymes, enzyme inhibitors and enzymes, aptamers, and the like. Furthermore, specific binding pairs can include members that are analogs of the original specific binding members, for example, an analyte-analog. Immunoreactive specific binding members include antigens, antigen fragments, antibodies and antibody fragments, both monoclonal and polyclonal, protein subunits and complexes thereof, including those formed by recombinant DNA molecules.

The term “substrate molecule” as used herein, refers to a molecule that interacts specifically with a reporter molecule. By “interacts specifically” it is meant that the substrate molecule exhibits essentially a structural, physical, conformational change leading to the generation of a measurable physical signal, or any equivalent or combination thereof.

The term “specificity” as used herein, refers to the ability of a binding moiety to bind preferentially or predominantly to a biomarker of a thyroid medical condition, as described herein, versus a different antigen, and does not necessarily imply high affinity (as defined further herein). A binding moiety that can specifically bind to and/or that has affinity for a specific biomarker of a thyroid medical condition is said to be “against” or “directed against” the antigen or antigenic determinant. A probing or recognition molecule according to the invention is said to be “cross-reactive” for two different biomarkers of a thyroid medical condition molecules if it is specific for both these different biomarkers of a thyroid medical condition.

The term “affinity”, as used herein, refers to the degree to which a probing molecule binds to a biomarker of a thyroid medical condition so as to shift the equilibrium of free biomarker of a thyroid medical condition toward the presence of a complex formed by their binding. Thus, for example, where a biomarker of a thyroid medical condition and a probing molecule are combined in relatively equal concentration, a probing molecule of high affinity will bind to the available biomarker of a thyroid medical condition so as to shift the equilibrium toward high concentration of the resulting complex. The dissociation constant (Kd) is commonly used to describe the affinity between the probing molecule and its target. In some embodiments, the dissociation constant is lower than: 10⁻² M, 10⁻³ M, 10⁻⁴ M, 10⁻⁵ M, 10⁻⁶ M, 10⁻⁷ M, 10⁻⁸ M, or 10⁻⁹ M, or any value and range therebetween. Each possibility represents a separate embodiment of the invention.

The terms “specifically bind” and “specific binding”, as used herein, refer to the ability of a binding domain to preferentially bind to a particular biomarker of a thyroid medical condition that is present in a homogeneous mixture of different molecules. In some embodiments, a specific binding interaction will discriminate between desirable and undesirable molecules in a sample, in some embodiments more than about 2- to 100-fold or more (e.g., more than about 1000- or 10,000-fold).

As used herein and in the claims, the term “functionalized surface” refers to a surface of an article that has been modified so that one or a plurality of molecules or functional groups are present thereon. In some embodiments, the plurality of molecules or functional groups are bound to the functionalized surface. The manner of treatment is dependent on, for example, the nature of the chemical compound to be synthesized and the nature and composition of the surface.

As used herein, the term “surface” refers to the material that the sections are made of. In some embodiments, surface is referred to an outer surface. A variety of materials can be used as surface according to the present invention. The materials include any material that can act as a support for attachment of the molecules of interest. Such materials are known to those of skill in this art. These materials include, but are not limited to, organic or inorganic polymers, natural and synthetic polymers, including, but not limited to, agarose, cellulose, nitrocellulose, cellulose acetate, other cellulose derivatives, dextran, dextran-derivatives and dextran co-polymers, other polysaccharides, glass, silica gels, gelatin, polyvinyl pyrrolidone, rayon, nylon, polyethylene, polypropylene, polybutylene, polycarbonate, polyesters, polyamides, vinyl polymers, polyvinylalcohols, polystyrene and polystyrene copolymers, polystyrene cross-linked with divinylbenzene or the like, acrylic resins, acrylates and acrylic acids, acrylamides, polyacrylamides, polyacrylamide blends, co-polymers of vinyl and acrylamide, methacrylates, methacrylate derivatives and co-polymers, other polymers and co-polymers with various functional groups, latex, butyl rubber and other synthetic rubbers, silicon, glass, paper, natural sponges, insoluble protein, surfactants, red blood cells, metals, metalloids, magnetic materials, or other commercially available media. In some embodiments, the surface comprises a water absorbing material, as described hereinabove.

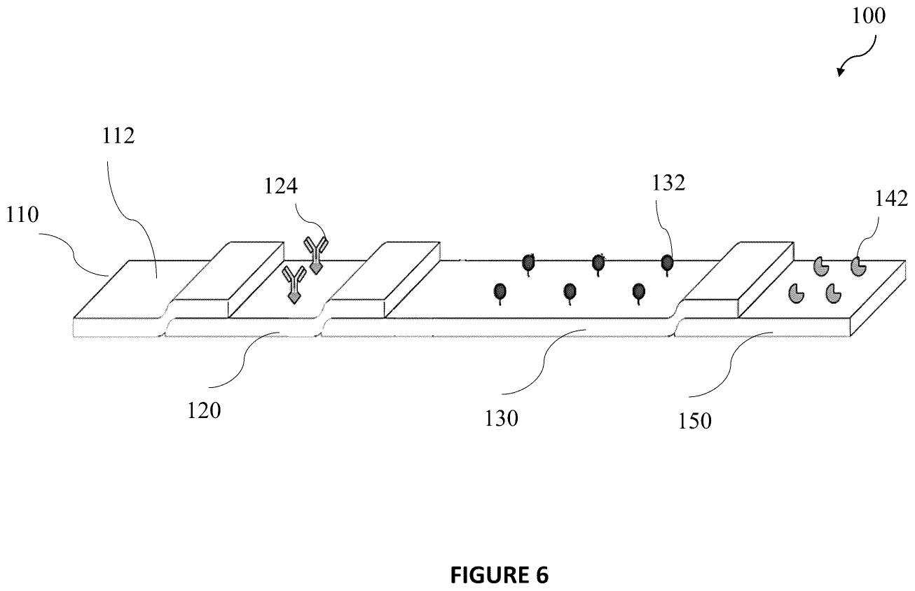

Reference is made to FIG. 6, which is a simplified illustration of some of the components of a device 100, according to some embodiments of the invention.

According to some embodiments of the present invention, section 1 110, section 2 120, section 3 130 and section 4 150 are arranged along a horizontal axis and in liquid communication allowing lateral flow from section 1 throughout all sections to section 4.

In some embodiments, section 1 110, section 2 120, section 3 130 and section 4 150 are in contact with each other, so as to allow a lateral flow from section 1 throughout all sections to section 4.

In some embodiments, section 1 110, section 2 120, section 3 130 and section 4 150 are partially overlapping. In some embodiments, overlapping is in the range of 0.01% to 99% of the total surface of a section. In some embodiments, section 1 is partially overlapping above section 2. In some embodiments, section 1 is partially overlapping bellow section 2. In some embodiments, section 2 is partially overlapping bellow section 3. In some embodiments, section 2 is partially overlapping above section 3. In some embodiments, section 3 is partially overlapping above section 4. In some embodiments, section 3 is partially overlapping bellow section 4.

In some embodiments, at least three sections of a device according to the present invention are disposed along more than one plane. In some embodiments, two consecutive sections are disposed along one or more planes. In some embodiments, section 1 110, section 2 120, section 3 130 and section 4 150 share at least one plane. In some embodiments, all sections are disposed along the same plane.

According to some embodiments of the present invention, section 1 110, section 2 120, section 3 130 and section 4 150 serve as solid support onto which different components are either adsorbed or immobilized (such as bound). In some embodiments, section 2 120, section 3 130 and section 4 150 comprise a surface in contact with or bound to the component (such as a substrate molecule, a probing molecule, or a biomarker of a thyroid medical condition), wherein the surface is as described hereinabove. In some embodiments, the component on section 2 120, comprise a probing molecule (such as an immunoreagent) adsorbed, in contact with or bound to the section. In some embodiments, the component on section 2 120, comprise the probing molecule adsorbed to the section. The component on section 3 130 comprise immunoreagents and are either adsorbed or covalently immobilized (e.g., covalently bound) to the section. In some embodiments, the different components are immobilized prior to the assembly of the sections. In some embodiments, the different components are immobilized after the assembly of the sections.

In some embodiments, section 1 110 comprises a sample collecting surface 112, section 2 120 comprises a surface comprising a probing-reporter molecule complex 124, section 3 comprises surface functionalized with a biomarker of a thyroid medical condition 132 and section 4 150 comprises a surface with a substrate molecule deposited thereon 142. In some embodiments, section 3 130 comprises a calibration area 140. In some embodiments, section 3 130, further comprises a calibration area 140 comprising a substrate molecule placed adjacent to section 2 120.

In some embodiments, section 1 110 comprises hydrogen peroxide (H₂O₂). In some embodiments, the invention comprises depositing H₂O₂ to section 1 110 of the device of the invention.

In some embodiments, section 1 110, section 2 120, section 3 130 and section 4 150 comprise a membrane. In some embodiments, a membrane comprises polyester. In some embodiments, a membrane comprises cellulose.

As used herein the term “membrane” refers to a boundary, a layer, barrier, or material, which may, or may not be permeable. The term “membrane” may further refer to an interface. In some embodiments, the terms “membrane” and “surface” are used herein interchangeably. Unless specified otherwise, membranes may take the form a solid, liquid, or gel, and may or may not have a distinct lattice, none cross-linked structure, or cross-linked structure. In some embodiments, the membrane is a fibrous membrane.

In some embodiments, section 1 110, section 2 120, section 3 130 and section 4 150, comprise a matrix. The matrix defines a lateral flow path. In some embodiments, the path is a microfluidic path. In some embodiments, the flow path is axial, and the flow is one way directed. In some embodiments, the flow direction is downstream from section 1. As used herein the term “downstream” refers a location to which liquid applied to the sample collecting surface will flow, such location being opposite direction to section 1. In some embodiments, the dissolved or dispersed components of the liquid sample are carried at substantially equal rates and with relatively unimpaired flow laterally through the matrix. In some embodiments, the lateral flow as used herein, refers to a capillary flow. In some embodiments, the lateral flow is generated by a capillary action. In some embodiments, the dissolved or dispersed components of the liquid sample are modulated by the added PVA membrane and other surface-active materials or ionic buffers forces.

Typical matrix materials that can be used in a device according to the present invention include high density polyethylene, polyvinyl chloride, polyvinyl acetate, copolymers of vinyl acetate and vinyl chloride, polyamide, polycarbonate, nylon, glass fiber, orlon, polyester, polystyrene, cotton, cellulose and the like, or blends. The optimum pore diameter for the membrane for use in the invention is about 20 μm to about 140 μm. Other materials, such as untreated paper, nitrocellulose, derivatized nylon, cellulose and the like may also be used according to the present invention.

In some embodiments, the matrix or the membrane comprises a hydrophilic material. In some embodiments, the hydrophilic material is a hydrophilic polymer. In some embodiments, the matrix or the membrane comprises a polymer wettable by an aqueous solution.

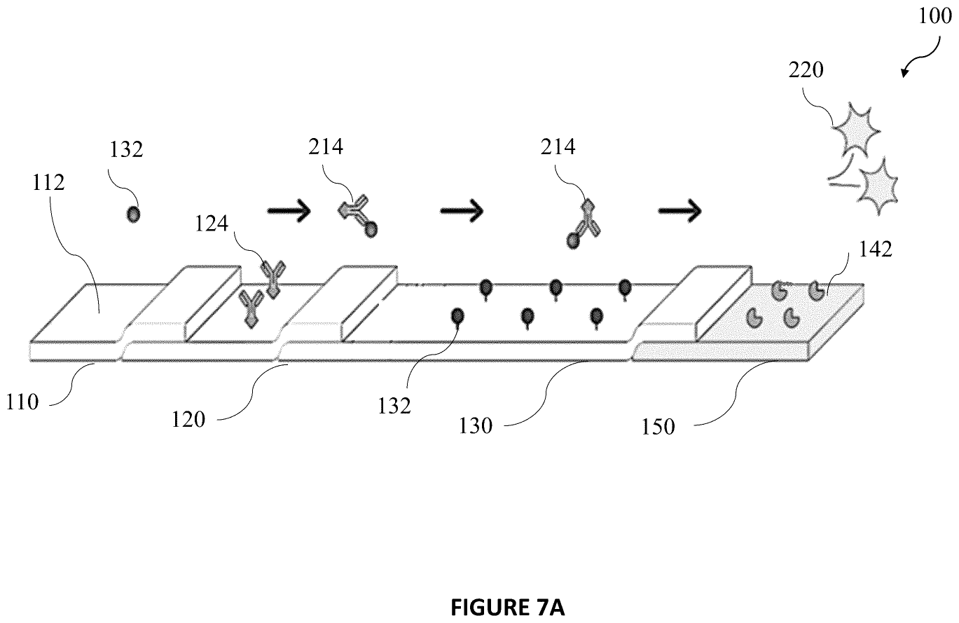

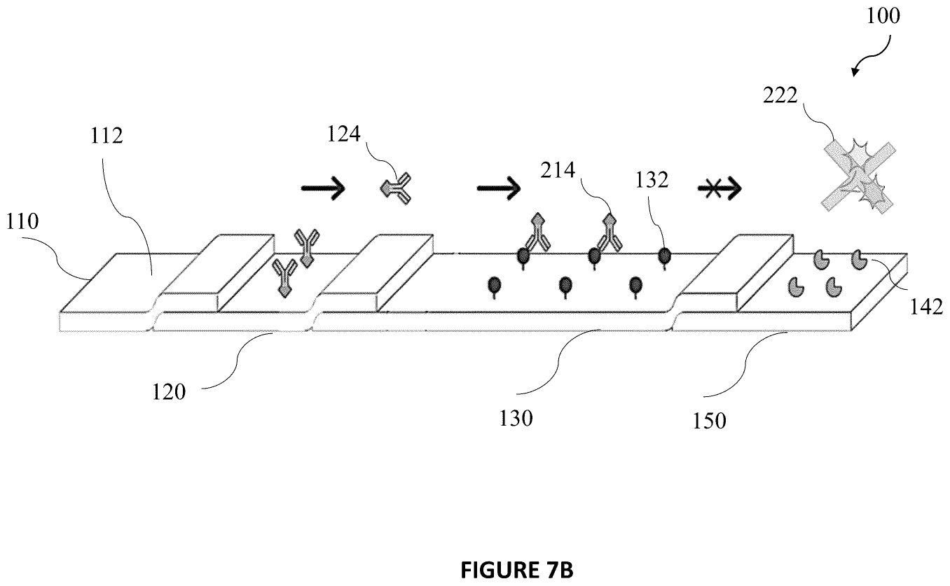

Reference is now made to FIG. 7, which is a simplified illustration of how a device 100 works during an assay measurement according to some embodiments of the present invention. In some embodiments, the device comprises a section 1 110 with a sample collecting surface 112, a section 2 120 comprising a surface with a probing molecule 124 linked to a reporter molecule, a section 3 comprising surface functionalized with a biomarker of a thyroid medical condition 132 and section 4 150 comprising a surface with a deposited substrate molecule 142.

There are two main possibilities that can happen during measurements. A first possibility is represented FIG. 7A. In some embodiments, a liquid sample with a target biomarker of a thyroid medical condition 132 is placed in section 1 110. The sample migrates to section 2 120, via lateral flow, where it encounters the probing molecule 124. A complex 214 is formed based on molecular recognition (such as affinity-based interaction or binding between the probing molecule and the biomarker of a thyroid medical condition), wherein complex 214 comprises the biomarker of a thyroid medical condition 132 bound or in contact with the probing molecule 124, and wherein the probing molecule 124 is bound to a reporter molecule generating a trigger. The biomarker of a thyroid medical condition-probing-reporter molecule complex 214 formed, continues to migrate via lateral flow to section 3 130 comprising the biomarker of a thyroid medical condition 132. Since the biomarker of a thyroid medical condition-probing molecule complex 214 was already formed, the complex 214 cannot be immobilized in section 3 130 and will continue and migrate to section 4 150 comprising a surface with the deposited substrate molecule 142. Here, the complex 214 or the trigger generated by the reporter molecule will interact with the substrate molecule 142. The interaction 218 formed will result in a reaction, thereby generating a signal 220, and confirming the presence of the biomarker of a thyroid medical condition in the sample in an absolute manner. The type of signal generation will depend on the reporter molecule used that is conjugated to the reporter molecule and the substrate molecule deposited in section 4 150.

Reference is now made to FIG. 7B. In some embodiments, in the case of a sample without a target biomarker of a thyroid medical condition 132, the sample migrates from section 2 120, via lateral flow, where it encounters the probing molecules 124. The reporter molecules will then migrate unbound with the sample to section 3 130, where they will be linked to the biomarker of a thyroid medical condition 132, forming the complex 214 and stopped from migrating further to the next section 4 150, thus no visible signal will be observed in section 4 150 (as exemplified in FIG. 7B by the cross 222).

In some embodiments, section 1 110, section 2 120, section 3 130 and section 4 150, are arranged in such way that section 3 130 is able to receive both biomarker of a thyroid medical condition-probing-reporter molecule complex 214 and excess of free reporter molecules 124 and section 4 150 is able to receive only biomarker of a thyroid medical condition-probing-reporter molecule complex 214. In some embodiments, section 3 130 comprising a biomarker of a thyroid medical condition 132, positioned between section 2 120 and section 4 150, ensures that only biomarker of a thyroid medical condition-probing-reporter molecule complex 214 migrates to section 4 150.

In some embodiments, section 1 110, section 2 120, section 3 130 and section 4 150, are in liquid communication, allowing lateral flow from section 1 to section 2, from section 2 to section 3, and from section 3 to section 4.

### Calibration Area

In some embodiments, a device according to the present invention further comprises a calibration area.

In some embodiments, a device according to the present invention further comprises a calibration area positioned between section 2 and section 3 and comprising a substrate. In some embodiments, calibration area is in liquid communication with or is coupled to section 2 and section 3.

In some embodiments, a device according to the present invention further comprises a calibration area positioned between section 4 and section 5 and comprising a substrate. In some embodiments, calibration area is in liquid communication with or is coupled to section 4.

In some embodiments, the calibration area is disposed in section 3. In some embodiments, the device of the invention comprises a plurality of calibration areas. In some embodiments, a plurality comprises at least 2. In some embodiments, a plurality of calibration areas are disposed in section 3. In some embodiments, a calibration area is disposed in section 3 in a position closer to the contact point of section 3 with section 2, compared to the contact point of section 3 with section 4. In some embodiments, a calibration area is disposed in section 3 in a position closer to the contact point of section 3 with section 4, compared to the contact point of section 3 with section 2. In some embodiments, the plurality of calibration areas are disposed such that section 3 comprises at least a first calibration area in a position closer to the contact point of section 3 with section 2, compared to the contact point of section 3 with section 4 and at least a second calibration area in a position closer to the to the contact point of section 3 with section 4, compared to the contact point of section 3 with section 2. In some embodiments, the calibration area is disposed in section 4.

In some embodiments, a device comprising sections 1 to 4, comprises a plurality of calibration areas in section 3, as disclosed herein.

In some embodiments, a device comprising section 1 to 5, comprises a calibration area in section 4, as disclosed herein.

In some embodiments, a plurality of signals are generated in a plurality of calibration areas and are compared to one another. In some embodiments, a relative amount of signal is provided by determining the amount of signal in at least a first calibration area of the plurality of calibration areas, determining the amount of signal in at least a second calibration area of the plurality of calibration areas, and determining the ratio between them.

In some embodiments, the at least first calibration area of the plurality of calibration areas provides a reference point and may be considered as a “positive control”.

In some embodiments, the at least second calibration area of the plurality of calibration areas provides a signal equivalent to the unknown amount of the analyte.

In some embodiments, the device and assay disclosed are used to determine the unknown amount of an analyte, as described herein, e.g., a thyroid related-condition biomarker, by comparing the amount of signal generated in the at least second calibration area of the plurality of calibration areas to the at least first calibration area of the plurality of calibration areas.

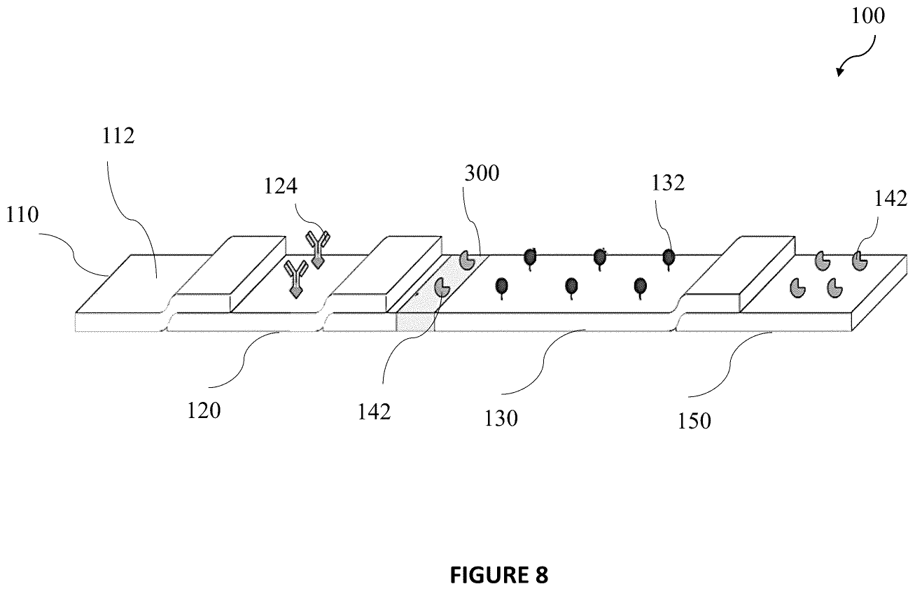

Reference is now made to FIG. 8, which is a simplified illustration of a device 100 according to the present invention comprising a calibration area 300, according to some embodiments of the invention.

In some embodiments, a calibration area 300 comprising a substrate molecule 142 is placed adjacent to section 2 120. In some embodiments, a calibration area 300 is in liquid communication with or coupled to section 2 120.

There are two main possibilities that can happen during measurements in a device 100 comprising a calibration area 300, according to some embodiments, of the invention.

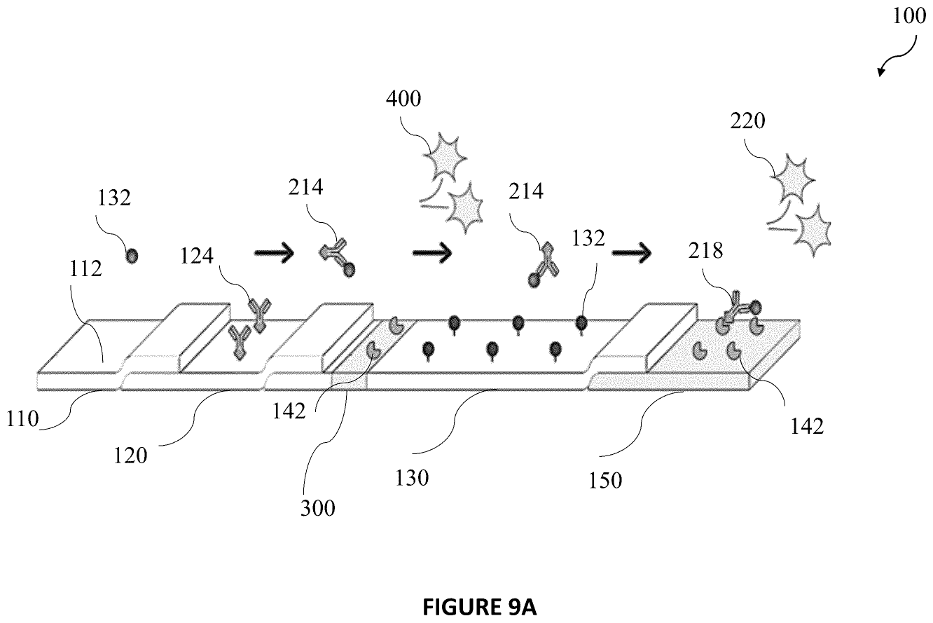

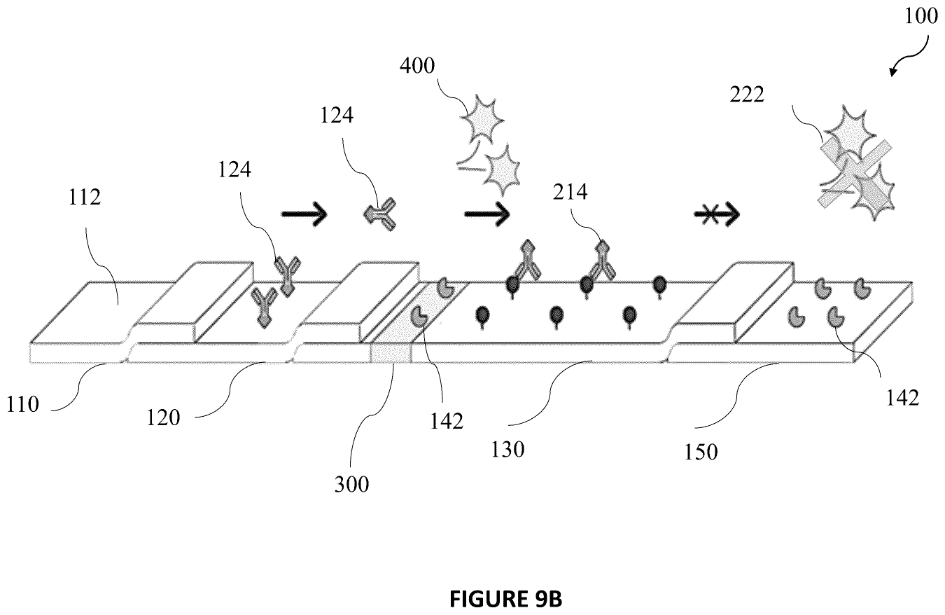

A first possibility is represented by FIG. 9A. In some embodiments, a liquid sample with a target biomarker of a thyroid medical condition 132 is placed in section 1 110. The sample migrates to section 2 120, via lateral flow, where it encounters the probing molecules 124. A complex 214 is formed based on molecular probing. The biomarker of a thyroid medical condition-probing-reporter molecule complex 214 formed, continues to migrate via lateral flow to calibration area 300 comprising a substrate molecule 142. The substrate molecule 142 is able to produce a first signal 400 when oxidized by hydrogen peroxide which is catalyzed by the reporter in the biomarker of a thyroid medical condition-probing-reporter molecule complex 214 and if present, the reporter in the probing-reporter molecule complex 124, thereby indicating the proper function and total quantity of the reporter molecule 124 and the reported molecule 214 entering section 3 130. The signal 400 produced, can also be used for signal calibration. Signal calibration can be done by measuring (using photodetector, cellphone, potentiostat (electrochemical signal, fluorescent measuring device, camera) and correlating signal intensity with concentration of a probing-reported molecule complex entering section 3 130.

The biomarker of a thyroid medical condition-probing-reporter molecule complex 214 formed, continue in a lateral flow, is not able to bind to the biomarker of a thyroid medical condition 132 immobilized in section 3 130, and therefore will continue and migrate to section 4 150 comprising a surface with the deposited substrate molecule 142, while migrating is through the line of substrate at the end portion of section 3 (e.g., resulting in a positive signal at this position). The complex, containing reporter oxidizes the substrate molecule thereby triggering a reaction and generating a second signal 220. The type of signal generation will depend on the signal molecule used (reporter molecule and substrate molecule).

Reference is now made to FIG. 9B. In some embodiments, in the case of a sample without a target biomarker of a thyroid medical condition 132, the sample migrates from section 2 120, via lateral flow, where it encounters the probing-reporter molecules 124. The probing-reporter molecules will then migrate unbound with the sample to calibration area 300 as described herein. Also, in this case, a signal 400 is generated, thereby indicating the proper function and quantity of the probing-reporter molecule 124. The probing-reporter molecules will continue to migrate to section 3 130, where they will be linked to the biomarker of a thyroid medical condition 132 and stopped from migrating further to the next section 4 150, thus no visible signal will be observed in section 4 150 (as exemplified in FIG. 9B by the cross on top of the signal 222).

In some embodiments, the calibration area 300 is designed to receive both biomarker of a thyroid medical condition-probing-reporter molecule complex 214 and excess of free probing-reporter molecules 124. In some embodiments, the calibration area 300 is designed to receive a trigger generated by the reporter molecule, so as to form a detectable signal. In some embodiments, section 4 150 is designed to receive only biomarker of a thyroid medical condition-probing-reporter molecule complex 214. In some embodiments, section 4 150 is designed to receive only the trigger generated by the reporter molecule. In some embodiments, section 4 150 is designed to receive only probing-reporter molecule complex 124.

In some embodiments, section 1 110, section 2 120, section 3 130 and section 4 150, are arranged in such way that section 3 130 is able to receive both biomarker of a thyroid medical condition-probing-reporter molecule complex 214 or the trigger and excess of free probing-reporter molecules 124 and section 4 150 is able to receive only biomarker of a thyroid medical condition-probing-reporter molecule complex 214 or the trigger. In some embodiments, section 3 130 comprising a biomarker of a thyroid medical condition 132, positioned between section 2 120 and section 4 150, ensures that only biomarker of a thyroid medical condition-probing-reporter molecule complex 214 or the trigger migrates to section 4 150.

In some embodiments, section 1 110, section 2 120, section 3 130 and section 4 150, are in liquid communication.

In some embodiments, a device as described herein comprises calibration area comprising a substrate molecule, wherein the calibration area is placed adjacent to section 2. In some embodiments, a device as described herein comprises calibration area comprising a substrate molecule placed between section 2 and section 3. In some embodiments, the calibration area comprises a membrane, wherein the membrane is as described hereinabove.

In some embodiments, the calibration area is placed before the surface functionalized with a biomarker of a thyroid medical condition. In some embodiments, the calibration area is devoid of the biomarker of a thyroid medical condition. In some embodiments, the calibration area is devoid of a probing molecule. In some embodiments, the calibration area is devoid of a reporter molecule. In some embodiments, when the substrate molecule of the calibration area encounters a reporter molecule, the reporter molecule generates a trigger, that upon interaction with the substrate molecule generates a signal giving an indication for the functionality and quantity of the reporter molecule and a reference of total signal intensity. In some embodiments, the signal intensity is used for signal calibration.

In some embodiments, the substrate molecule is in contact with the surface of the calibration area. As used herein, the term “in contact” may be referred to bound via a covalent or a non-covalent bond. In some embodiments, a substrate molecule is a color producing signal substrate molecule such as 3,3′-Diaminobenzidine (DAB), 5-Bromo-4-Chloro-3-IndolylPhosphate (BCIP), 3,3′,5,5′-tetramethylbenzidine (TMB), p-Nitrophenyl Phosphate, Disodium Salt (PNPP), 2,2′-Azinobis [3-ethylbenzothiazoline-6-sulfonic acid]-diammonium salt (ABTS), o-phenylenediamine dihydrochloride (OPD). In some embodiments, a substrate molecule is a light producing signal substrate molecule such as 1,2-Dioxetane (CDP-star and CSPD).

In some embodiments, the substrate molecule is deposited on the or to the calibration area along with a signal enhancer. In some embodiments, a signal enhancer comprises any compound or agent suitable for enhancing the selectivity and/or sensitivity of a signal generated by the device and method of the invention.

In some embodiments, the signal enhancer comprises or consists of imidazole.

### Section 1

In some embodiments, a device as described herein comprises a section 1, comprising a sample collecting surface.

In some embodiments, a collecting surface is a filter. In some embodiments, a collecting surface is a solid support that may hold the sample. In some embodiments, a collecting surface comprises a membrane or matrix, wherein the membrane or matrix is as described hereinabove.

In some embodiments, a collecting surface comprises a material capable of absorbing or adsorbing a liquid sample.

In some embodiments, the size and shape of section 1 may vary.

In some embodiments, the sample collecting surface is comprised of filter for whole cell and large bodies filtration.

In some embodiments, the sample collecting surface comprises a buffer for controlling pH and ionic strength. In some embodiments, the collecting surface comprises CoCl₂. In some embodiments, the collecting surface comprises nickel. In some embodiments, any one of CoCl₂, nickel, H₂O₂, and any combination thereof is deposited to the collecting surface.

As used herein the term “sample collecting surface” refers to a surface wherein the sample is applied. The applied sample migrates consecutively from the sample collecting surface in section 1 to section 2, section 3, and section 4 in this specific order.

### Section 2

In some embodiments, a device as described herein comprises a section 2, comprising a surface comprising a probing molecule linked or bound to a reporter (signal) molecule. In some embodiments, section 2, comprises a surface comprising a deposited probing molecule linked to a reporter (signal) molecule. In some embodiments the probing molecule has specific affinity to the biomarker of a thyroid medical condition. In some embodiments, the reporter molecule generates a chemically and/or an electric and/or a fluorescent and/or a physically detectable reaction. In some embodiments, the reporter molecule generates a trigger. In some embodiments, the probing molecule is dried on the surface of section 2. In some embodiments, the probing molecule is unbound to the surface of section 2. In some embodiments, the trigger induces a signal formation upon contacting the substrate molecule. In some embodiments, the trigger is capable to interact chemically (e.g., via a reaction and/or a non-covalent binding), physically (e.g., via photon-induced excitation, via interactions with ionizing radiation, or by inducing electromagnetic field-based interaction). In some embodiments, the trigger comprises at least one of: a reactive compound (such as a peroxide, or any compound capable of reacting with the substrate molecule so as to generate a signal), an electromagnetic radiation, an ionizing radiation, and a charged particle or a combination thereof. In some embodiments, the trigger is a photon having a wavelength sufficient to induce a fluorescence, a luminescence, a phosphorescence or a colorimetric reaction of the substrate molecule.

The term “probing molecule” as used herein refers to a molecule possessing a high affinity to (i.e., an equilibrium dissociation constant values of K_{d}≤10⁻⁹ M), in a biologically relevant system (e.g., in vitro, ex vivo or in vivo). In some embodiments, the “probing molecule” comprises a “reporter molecule”. In some embodiments, the “probing molecule” comprises a “reporter molecule” which is capable of generating and generates a measurable signal detectable by external means.

The term “reporter molecule” as used herein refers to a chemical group or a molecular motif possessing medium to high affinity towards a molecular reagent or a biomolecule that induces or mediates a reaction that yields a product, that can be monitored instrumentally. In some embodiments, “reporter molecule” include chromogens, catalysts such as enzymes, luminescent compounds such as fluorescein and rhodamine, chemiluminescent compounds such as dioxetanes, acridiniums, phenanthridiniums and luminol, radioactive elements, electroactive compounds, TEMPO, 1,4,5,8-naphthalenetetracarboxylic diimide (NTCDI), and direct visual labels. The selection of a particular reporter molecule is not critical, but it will be capable of producing a signal either by itself or in interaction with one or more additional substances.

Examples of reporter enzymes which can be used to practice the invention include hydrolases, lyases, oxidoreductases, transferases, isomerases and ligases. Some preferred examples are glucose oxidase, phosphatases, esterases, glycosidases and peroxidases. In some embodiments, the reporter molecule is selected from the group consisting of protein, enzyme, horseradish peroxidase, nucleotide, dye, quantum dot, fluorophores, gold, silver and platinum. In some embodiments, the reporter molecule generates a chemically active trigger such as hydrogen peroxide, which oxidizes the substrate molecule.

In some embodiments, a reporter molecule is selected from an enzyme, luminescent substrate compound, fluorescent, electrochemical active compound, fluorophores (organic, quantum dots, fluorescent proteins), organic dye, magnetic particles, gold particles.

In some embodiments, a probing molecule is selected from DNA, proteins, antigen, bioreceptors, aptamers, phage displayed epitopes, biomimetics, peptide, nucleic acid or antibodies linked to some reporters.

In some embodiments, the recognition element at section 120 is allowed to flow with the sample. The substrate molecule (both on section 400 and 150), and capture element stay in place during the assay time.

### Section 3

In some embodiments, a device as described herein comprises a section 3 comprising a surface functionalized with a biomarker of a thyroid medical condition or equivalent thereof, wherein the surface is as described hereinabove. In some embodiments, the biomarker of a thyroid medical condition or equivalent thereof is bound to the surface of section 3.

In some embodiments, if a sample without a biomarker of a thyroid medical condition is used, during the migration, of the sample, the excess of free probing-reporter molecules will be conjugated into the section 3 functionalized with the biomarker of a thyroid medical condition or equivalent thereof and will not migrate further to section 4.

In some embodiments, if a sample with a biomarker of a thyroid medical condition is used, since the biomarker of a thyroid medical condition-probing-reporter molecule complex is formed before section 3, the sample will continue and migrate to section 4 comprising a surface with a deposited substrate molecule, thereby generating a signal. The type of signal generation will depend on the reporter molecule used that is conjugated to the probing molecule and/or the substrate molecule deposited.

In some embodiments, an equivalent to the biomarker of a thyroid medical condition is used. In some embodiments, an equivalent to the biomarker of a thyroid medical condition refers to an analogous molecule. An equivalent to the biomarker of a thyroid medical condition is a molecule with interaction to the same active site on the probing molecule. In some embodiments, a biomarker of a thyroid medical condition analog can be a synthetic peptide or a peptide-displaced phage or a subunit of a protein.

### Section 4

In some embodiments, a device according to the present invention, comprises a section 4 comprising a surface in contact or bound to a substrate molecule, wherein the surface is as described hereinabove. In some embodiments, section 4 comprises the same substrate molecule as present in the calibration area as described elsewhere herein.

In some embodiments, section 4 comprises a surface comprising electrodes. In some embodiments, section 4 comprises a surface in contact with or bound to a substrate molecule selected from a fluorophore, a luminophore, a photoluminophore, a radioluminescent material, and a light-reactive material or a combination thereof. In some embodiments, the substrate molecule comprises a molecule capable of reacting with peroxide, so as to form a detectable signal.

In some embodiments, when the substrate molecule of section 4 encounters a reporter molecule it is able to emit a signal with a certain intensity. In some embodiments, the signal intensity is compared to the signal obtained from calibration area and used for signal calibration. In some embodiments, the signal obtained is proportional to the biomarker of a thyroid medical condition concentration in the sample. In some embodiments, the signal obtained is proportional to the biomarker of a thyroid medical condition concentration in the sample and the time from the sample reaching section 4 to the time of measurement. In some embodiments, the ratio between the signal obtained from the calibration area and section 4, is used as internal standard.

In some embodiments, the signal ratio between the calibration area and section 4, can be compared to predetermined values and can indicate the amount of a biomarker of a thyroid medical condition present in a sample. In first encounter of the reporter to first substrate molecule zone, a signal proportional to the number of reporter molecules passing by is obtained. Thereafter when those reporter-biomarker of a thyroid medical condition complexes do not bind to the capture layer, they reach the second area with substrate molecule at the far end and again the reporter marker will oxidize those substrate molecules anew. In case very little biomarker of a thyroid medical condition is present then first signal is much higher than downstream signal. In case of saturation (or close to saturation of) reporter molecule to biomarker of a thyroid medical condition then the upstream and downstream signals are close to unit.

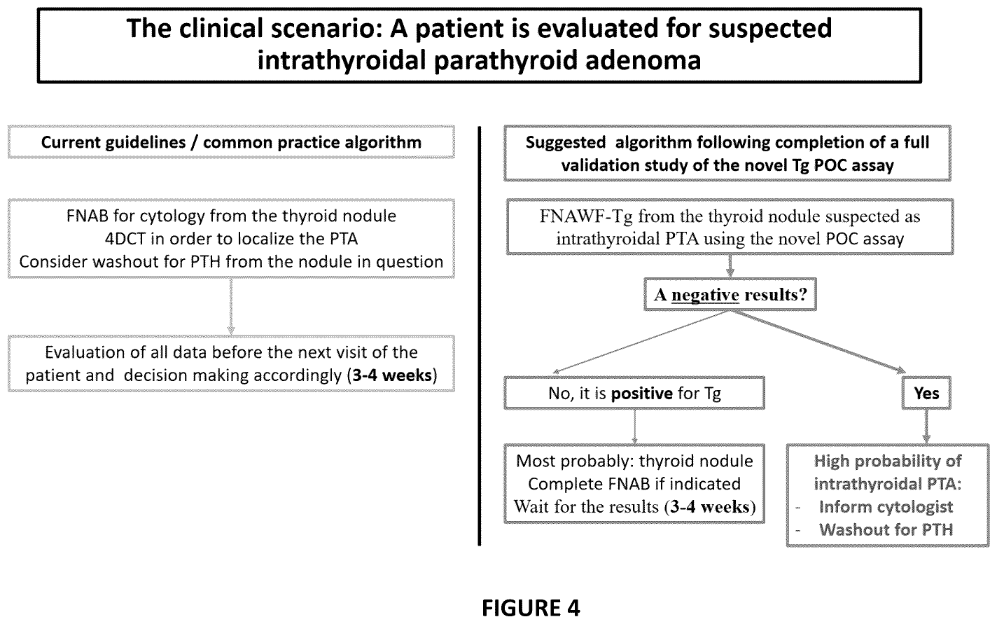

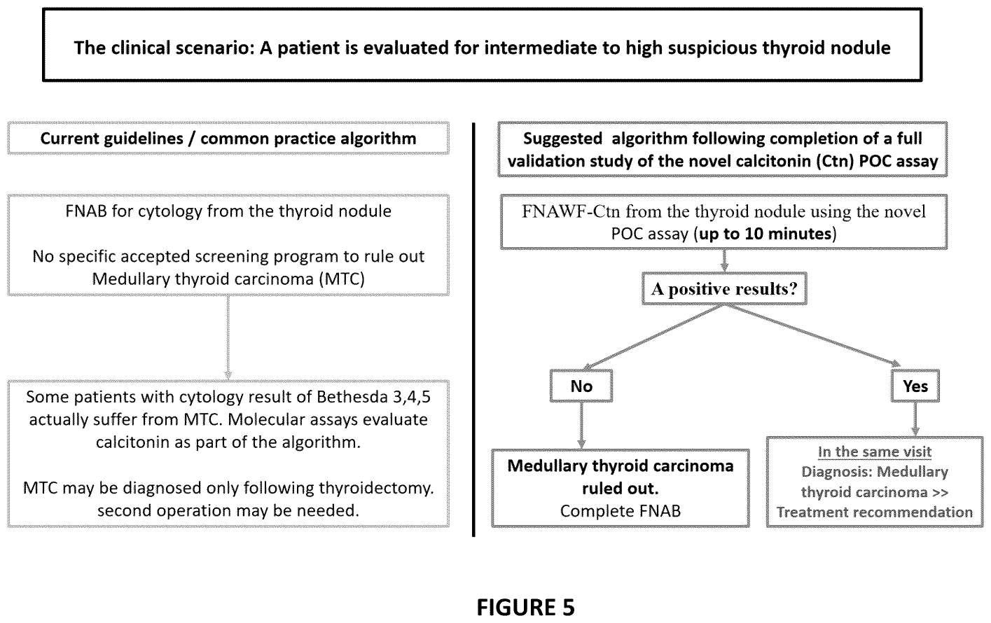

Referring to FIG. 4, in some embodiments, when there is no biomarker of a thyroid medical condition in the sample, the signal ratio between section 4 and the calibration area value is 0. In some embodiments, when there is a biomarker of a thyroid medical condition in the sample, the signal ratio between section 4 and the calibration area value is increasing in correlation with biomarker of a thyroid medical condition concentration and time from when the sample reach section 4.

In some embodiments, when there is no biomarker of a thyroid medical condition in the sample, the signal ratio between section 4 and the calibration area value is 0. In some embodiments, when there is a biomarker of a thyroid medical condition in the sample, the signal ratio between section 4 and the calibration area value is between 0 and 1 in correlation to biomarker of a thyroid medical condition concentration in the sample creating a relative signal.

In some embodiments, the substrate molecule is a colorimetric agent. In some embodiments, the substrate molecule is capable of reacting with the trigger (such as a peroxide) to result in color change. In some embodiments, the substrate molecule is a color producing substrate molecule such as 5-Bromo-4-Chloro-3-IndolylPhosphate (BCIP) or 3,3′,5,5′-tetramethylbenzidine (TMB), 3,3′-Diaminobenzidine (DAB), chromogenic.

In some embodiments, the substrate is DAB.

In some embodiments, the substrate, such as DAB, is further mixed or formulated with at least one additional agent or compound.

In some embodiments, the at least one additional compound or agent improves at least one activity associated with the substrate. In some embodiments, the at least one activity is selected from: solubility, loadability, migration, catalytic processability or enzymatic breakdown, or any combination thereof, according to the method and devices described herein.

In some embodiments, the at least one additional compound or agent increases production, amplification, intensity, or any combination thereof, or a signal generated by the enzymatic breakdown of the substrate.

In some embodiments, the at least one additional compound or agent reduces the migration of a substrate in the device as disclosed herein.

In some embodiments, the at least one additional compound or agent reduces the level of smearing of a generated by the enzymatic breakdown of the substrate.

In some embodiments, the at least one additional compound or agent is selected from: imidazole, polyvinyl alcohol (PVA), polyethylene glycol (PEG), or any combination thereof.

In some embodiments, PEG comprises PEG 20K, PEG 8K, or a combination thereof.

In some embodiments, the type of signal depends on the chosen reporter molecule and/or substrate molecule.

In some embodiments, signal detection, quantification or both is done using a reader or detection unit. In some embodiments, the device of the invention further comprises a detection unit. In some embodiments, the detection unit is in operable communication with the device. In some embodiments, the detection unit is in operable communication with section 4. In some embodiments, the detection unit is configured to detect the signal generated by the substrate molecule. In some embodiments, the detection unit comprises electric circuitry.

As used herein, the term “detection unit” refers to an instrument capable of detecting and/or quantitating data, such as on the sections described herein. The data may be visible to the naked eye but does not need to be visible. In some embodiments, the detection unit is in operable communication with a processor. A processor is of a general purpose computer, special purpose computer, or other programmable data processing apparatus to produce a machine, such that the instructions, which execute via the processor of the computer or other programmable data processing apparatus, create means for implementing the functions of the device. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the device. In some embodiments, the signal received form the device is processed by a software so as to generate an output, such as a positive or a negative reporting.

In some embodiments, the program code is excusable by a hardware processor.

In some embodiments, the hardware processor is a part of the control unit.

In some embodiments, there is further provided a read-out of the assay carried out in the device may be detected or measured using any suitable detection or measuring means known in the art. The detection means may vary depending on the nature of the read-out of the assay. In some embodiments, disclosed device also relates to an apparatus including the device in any embodiments thereof, and a detection unit as described herein.

In some embodiments, the detection unit provides a positive reporting. In some embodiments, the detection unit provides a negative reporting. As used herein “positive reporting” refers to an increase in the detection signal with the increase of biomarker of a thyroid medical condition concentration. As used herein the term “negative reporting” refers to no detection signal.

In some embodiments, a reader is an electrochemical detection unit. In some embodiments, a reader is a colorimetric detection unit. In some embodiments, a detection unit comprises a photodetector such as PhotomultiplierTubes (PMTS), CCD camera or complementary MOS (CMOS). In some embodiments, a detection unit is a cellphone. In some embodiments, a detection unit will include light source for excitation of a fluorescent reporter molecule and a photo detector. In some embodiments, a detection unit is a human.

In some embodiments, a signal is a color change. In some embodiments, a signal is light generation. In some embodiments, a signal is an electron flow. In some embodiments, a signal is an excited light source.

As used herein, the term “color” refers to the relative energy distribution of electromagnetic radiation within the visible spectrum. Color can be assessed visually or by using equipment, such as a photosensitive detector.

As used herein, the term “color change” refers to a change in intensity or hue of color or may be the appearance of color where no color existed or the disappearance of color.

In some embodiments, section 4 further comprises an active-pixel sensor (APS) or an electrode.

### Section 5

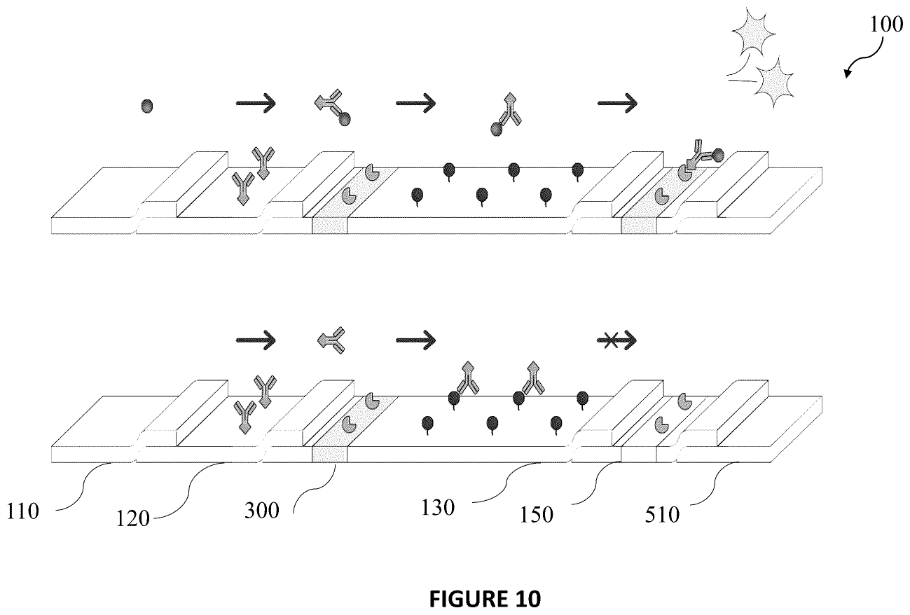

Reference is now made to FIG. 10. According to some embodiments of the present invention, the device further comprises a section 5. In some embodiments, section 5 is coupled to section 4 and in liquid communication with section 4. In some embodiments, there is provided a device comprising a section 1 110, section 2 120, section 3 130, section 4 150 and section 5 510 arranged along a horizontal axis and in liquid communication allowing lateral flow from section 1 throughout all sections to section 5.

In some embodiments, section 5 510 is devoid of reagents and is able to contain the whole sample volume.

Referring to FIG. 10, when the sample reach section 4 150, the reporter will interact with the substrate molecule to give a measurable signal, then continue in migration to section 5 510 to be absorbed.

In some embodiments, there are diffusible membranes between sections of the device, which modulate sample flow rate and interaction time between reagents during measurement procedure.

In some embodiments, a diffusible membrane is made of Polyvinyl Alcohol, paraffin.

In some embodiments, the device will be introduced to vibrations with frequency ranging between 0.1 kHz and 1,000 kHz, the vibration will encourage interactions between reagents and increase efficiency. In some embodiments, the vibrations are originating from an internal section. In some embodiments, the vibrations are originating from an external device.

In some embodiments, the flow can be modulated using a magnetic field.

In some embodiments, a device according to the present invention, is capable of detecting lower amounts of a biomarker of a thyroid medical condition in a sample when compared to a typical enzyme-linked immunosorbent assay (ELISA).

In some embodiments, a device according to the present invention detects the presence of a biomarker of a thyroid medical condition in a solution with a concentration lower than 25 ng mL⁻¹. In some embodiments, a device according to the present invention detects the presence of a biomarker of a thyroid medical condition in a solution with a concentration lower than 25 ng mL⁻¹, lower than 24 ng mL⁻¹, lower than 20 ng mL⁻¹, lower than 15 ng mL⁻¹, lower than 10 ng mL⁻¹, lower than 8 ng mL⁻¹, lower than 7 ng mL⁻¹, or lower than 5 ng mL⁻¹, including any value therebetween.

In some embodiments, the ratio of a reporter molecule in section 2 and a biomarker of a thyroid medical condition in section 3 is in the range of 1:1 to 1:20. In some embodiments, the ratio of a reporter molecule in section 2 and a biomarker of a thyroid medical condition in section 3 is in the range of 1:2 to 1:20, 1:3 to 1:20, 1:4 to 1:20, 1:8 to 1:20, 1:10 to 1:20, 1:12 to 1:20, or 1:15 to 1:20, including any range therebetween.

In some embodiments, the ratio of a reporter molecule in section 2 and a substrate molecule in calibration area is in the range of 1,000:1 to 1:1,000. In some embodiments, the ratio of a reporter molecule in section 2 and a substrate molecule in calibration area is in the range of 900:1 to 1:1000, 500:1 to 1:1,000, 300:1 to 1:1,000, 100:1 to 1:1,000, 50:1 to 1:1,000, 25:1 to 1:1,000, 1,000:1 to 1:900, 1,000:1 to 1:500, 1,000:1 to 1:300, 1,000:1 to 1:100, 1,000:1 to 1:50, or 1,000:1 to 1:25, including any range therebetween.

In some embodiments, the ratio of a reporter molecule in section 2 and a substrate molecule in section 4 is in the range of 1:1 to 1:1000. In some embodiments, the ratio of a reporter molecule in section 2 and a substrate molecule in section 4 is in the range of 1:1 to 1:900, 1:1 to 1:700, 1:1 to 1:500, 1:1 to 1:200, 1:1 to 1:100, 1:1 to 1:50, 1:1 to 1:25, or 1:1 to 1:10, including any range therebetween.

In some embodiments, the ratio of a substrate molecule in calibration area and a substrate molecule in section 4 is in the range of 1:1,000 to 1,000:1. In some embodiments, the ratio of a substrate molecule in calibration area and a substrate molecule in section 4 is in the range of 900:1 to 1:1,000, 500:1 to 1:1,000, 300:1 to 1:1,000, 100:1 to 1:1,000, 50:1 to 1:1,000, 25:1 to 1:1,000, 1,000:1 to 1:900, 1,000:1 to 1:500, 1,000:1 to 1:300, 1,000:1 to 1:100, 1,000:1 to 1:50, or 1,000:1 to 1:25, including any range therebetween.

In some embodiments, the device of the invention comprises at least 4 sections as described hereinbelow.

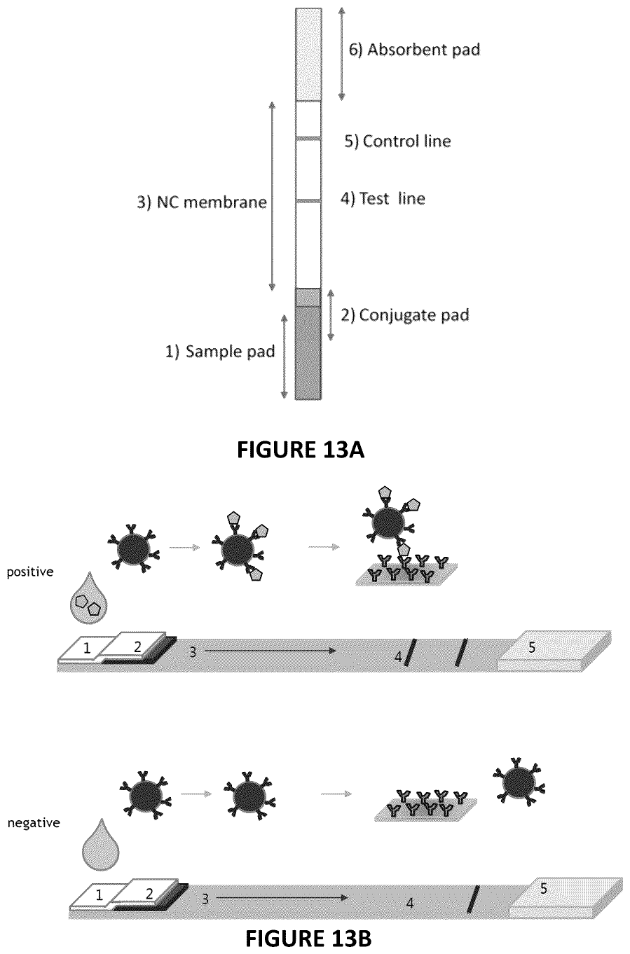

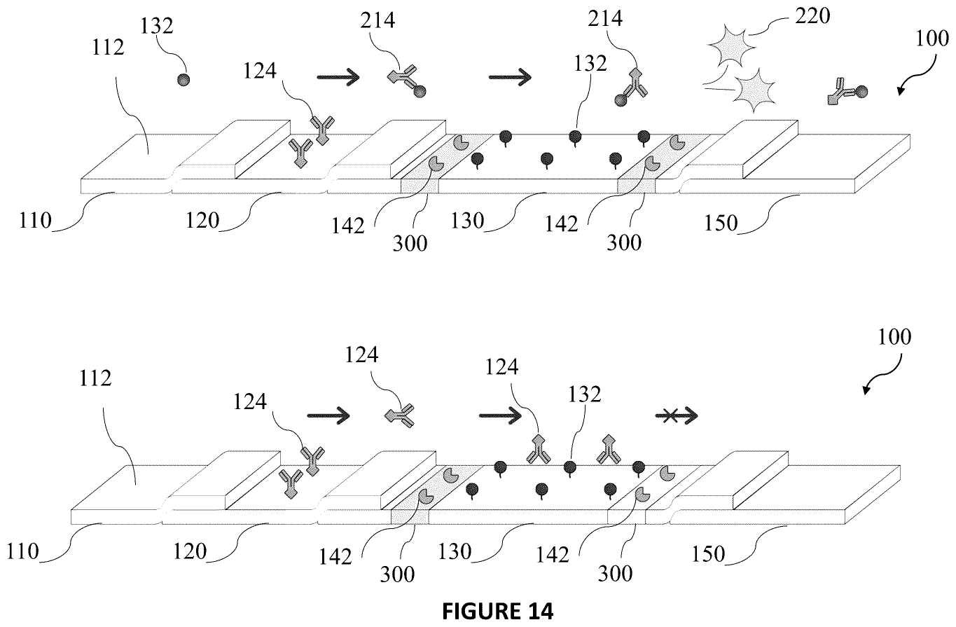

Reference is now made to FIG. 13. According to some embodiments, there is provided a device for rapid diagnosis of a thyroid medical condition comprising at least 4 sections comprising: section 1, section 2, section 3, and section 4, sequentially and linearly coupled to each other, wherein: section 1 comprises a sample collecting surface; section 2 comprises at least a first probing molecule having specific binding affinity to at least one biomarker, e.g., an analyte, of a thyroid medical condition being selected from the group consisting of: thyroglobulin, calcitonin, a parathyroid hormone, fragments thereof, and any combination thereof, wherein the at least first probing molecule is linked to a reporter molecule capable of generating a trigger; section 3 comprises a surface functionalized with at least a second probing molecule having specific binding affinity to at least one biomarker of a thyroid medical condition being selected from the group consisting of: thyroglobulin, calcitonin, a parathyroid hormone, fragments thereof, and any combination thereof; and section 4, wherein sections 1 to 4 are in liquid communication allowing flow of liquid sequentially from sections 1 to 4. In some embodiments, the device further comprises a section 5. In some embodiments, section 5 comprises or is an absorbent pad.

In some embodiments, section 1 comprises a sample collecting surface, as described herein.

In some embodiments, section 2 comprises a surface comprising a first probing molecule linked or bound to a reporter (signal) molecule. In some embodiments, section 2, comprises a surface comprising a deposited first probing molecule linked to a reporter (signal) molecule. In some embodiments the first probing molecule has specific affinity to the biomarker of a thyroid medical condition. In some embodiments, the reporter molecule generates a chemically and/or an electric and/or a fluorescent and/or a physically detectable reaction. In some embodiments, the reporter molecule generates a trigger. In some embodiments, section 2 comprises a reporter (signal) molecule linked to a control molecule. In some embodiments, the first probing molecule is dried on the surface of section 2. In some embodiments, the first probing molecule is unbound to the surface of section 2. In some embodiments, the trigger is a photon having a wavelength sufficient to induce a fluorescence, a luminescence, a phosphorescence or a colorimetric reaction of the substrate molecule. In some embodiments, a reporter molecule is selected from an enzyme, luminescent substrate compound, fluorescent, electrochemical active compound, fluorophores (organic, quantum dots, fluorescent proteins), organic dye, magnetic particles, gold particles, or any type of colored particles. In some embodiments, the first probing molecule e.g., an anti-thyroglobulin antibody, is linked to a particle. In some embodiments, the first probing molecule, e.g., an anti-thyroglobulin antibody is linked or chemically linked to a gold particle. In some embodiments, the trigger induces a signal formation upon contacting the substrate molecule. In some embodiments, the trigger is capable of interacting chemically (e.g., via a reaction and/or a non-covalent binding), physically (e.g., via photon-induced excitation, via interactions with ionizing radiation, or by inducing electromagnetic field-based interaction). In some embodiments, the trigger comprises at least one of: a reactive compound (such as a peroxide, or any compound capable of reacting with the substrate molecule so as to generate a signal), an electromagnetic radiation, an ionizing radiation, and a charged particle or a combination thereof.

In some embodiments, section 3 comprises a surface functionalized with a second probing molecule. In some embodiments, the first and the second probing molecule are targeting or capable of binding other regions of the biomarker of a thyroid medical condition or equivalent thereof. In some embodiments, the first and the second probing molecule are targeting or capable of binding other antigens of the biomarker of a thyroid medical condition or equivalent thereof. In some embodiments, the second probing molecule is immobilized to the surface of section 3. In some embodiments, the second probing molecule is immobilized to the surface of section 3 as defined in FIG. 13A “the Test line”.

In some embodiments, section 3 further comprises a third probing molecule being characterized by having specific binding affinity to the first probing molecule, as described herein. In some embodiments, the third probing molecule is characterized by having specific binding affinity to the first probing molecule linked to the reporter (signal) molecule. In some embodiments, the third probing molecule is characterized by having specific binding affinity to the first probing molecule bound to the biomarker of a thyroid (e.g., thyroglobulin) medical condition or equivalent thereof and linked to the reporter (signal) molecule. In some embodiments, the third probing molecule characterized by having specific binding affinity to the control molecule linked to a reporter (signal) molecule is immobilized to the surface of section 3. In some embodiments, the third probing molecule characterized by having specific binding affinity to the substrate molecule linked to a reporter (signal) molecule is immobilized to the surface of section 3 as defined in FIG. 13A “the Control line”.

In some embodiments, a signal as disclosed herein, is generated where a complex is formed between the lateral flowing analyte and an immobilized probing molecule. In some embodiments, a signal as disclosed herein, is generated where a complex is formed between the lateral flowing analyte bound to the first probing molecule and the immobilized second probing molecule (e.g., at the “test line”).

In some embodiments, a signal as disclosed herein, is generated where a complex is formed between a lateral flowing first probing molecule bound to an analyte, and the immobilized second probing molecule, e.g., at the “test line”.

In some embodiments, a signal as disclosed herein, is generated where a complex is formed between a lateral flowing first probing molecule, bound or unbound to an analyte, and the immobilized third probing molecule, e.g., at the “control line”.

In some embodiments, section 4 is devoid of reagents. In some embodiments, section 4 is able to contain the whole sample volume. In some embodiments, section 4 is capable of generating capillary flow.

In some embodiments, section 5 is devoid of reagents. In some embodiments, section 5 is able to contain the whole sample volume. In some embodiments, section 5 is capable of generating capillary flow.

Reference is now made to FIG. 13B. According to some embodiments of the present invention, the device comprises section 1 to section 4.

In some embodiments, a sample is applied in section 1. In some embodiments, section 2 (e.g., a conjugation pad) comprises a particle, e.g., a gold nanoparticle, comprising or linked to an anti-thyroglobulin antibody. In some embodiments, section 3 comprises a membrane allowing capillary flow, e.g., a nitro cellulose membrane. In some embodiments, the “test line” of section 3, comprises a second probing molecule, capable of specific binding with the analyte, e.g., a second type of an anti-thyroglobulin antibody. In some embodiments, section 3 comprises a “control line”. In some embodiments, the “control line” of section 3, comprises a third probing molecule, capable of specific binding with the first probing molecule, e.g., an antibody characterized by specific binding affinity to an anti-thyroglobulin antibody. In some embodiments, section 4 comprises an absorbent pad.

Reference is now made to FIG. 13B upper panel. In some embodiments, when a sample comprises a target thyroglobulin (positive), after deposition on section 1, the thyroglobulin connects to or is bound by the anti-thyroglobulin antibody conjugated to the gold nanoparticle to form a complex. The complex migrates via capillary force on the membrane towards the test line and control line. The formation of a visible test line indicates a positive sample (e.g., comprise an analyte), and results from the formation of a sandwich complex (thyroglobulin bound to two the two types of primary anti-thyroglobulin antibodies, e.g., the first and second probing molecules described herein). Further, the formation of a visible control line, indicates a proper assay is performed (e.g., a biological sample is provided, capillary flow is obtained, conditions for signal determination are provided, etc.) and results from the formation of a complex between the first probing molecule and the third probing molecule (e.g., between the anti-thyroglobulin antibody conjugated to the particle, e.g., a gold nanoparticle, and an anti-anti-thyroglobulin antibody). Other fluids and/or nonspecific materials continue to move to section 4, e.g., the absorbent pad.

Reference is now made to FIG. 13B lower panel. In some embodiments, when a sample devoid of an analyte is provided (e.g., a negative sample), the first and second probing molecules (e.g., antibodies) do not form a sandwich complex with an analyte (e.g., due to the absence of thyroglobulin), and thus continue to migrate pass the test line to section 4. In some embodiments, in a sample devoid of an analyte, a visible signal is formed in the control line due to the complexation of the first probing molecule and the third probing molecule. In some embodiments, in a sample devoid of an analyte, a visible signal is formed only in the control line due to the complexation of the first probing molecule and the third probing molecule.

### Method of Diagnosing Metastatic DTC

According to some embodiments, there is provided a method for diagnosing metastatic differentiating thyroid carcinoma (DTC) in a subject.

In some embodiments, the method comprises the steps of: (a) providing a sample comprising an extra thyroidal tissue or a fragment thereof derived from the subject; and (b) loading the sample from step (a) to the herein disclosed device, and detecting a signal produced by the substrate molecule.

In some embodiments, the detection of a signal is indicative of a presence of a biomarker of DTC in the sample. In some embodiments, the presence of the biomarker of DTC in the sample is indicative of a cancerous thyroidal cell being present in the sample, thereby diagnosing metastatic DTC in the subject.

In some embodiments, the lack of a detectable signal (e.g., negative detection, no detection, or any equivalent thereof) is indicative of an absence of a biomarker of DTC in the sample. In some embodiments, the absence of the biomarker of DTC in the sample is indicative of the absence of a cancerous thyroidal cell in the sample or that a cancerous thyroidal cell is absent from the sample, thereby determining the subject is not afflicted with metastatic DTC or the subject is metastatic DTC-free.

In some embodiments, DTC comprises papillary thyroid carcinoma (PTC), follicular thyroid carcinoma (FTC), or a combination thereof.

In some embodiments, the method is directed to diagnosing metastatic DTC in a sample derived or obtained from the subject. In some embodiments, the method is directed to in vitro or ex vivo diagnosing of DTC.

It would be apparent to a person of ordinary skill in the art that diagnosing in-vitro or ex vivo is performed in a tube, a plate, or any equivalent thereof, and not in a subject's body.

In some embodiments, the sample is devoid of a thyroid tissue. In some embodiments, the sample does not comprise a thyroid tissue. In some embodiments, a thyroid tissue is absent from the sample. In some embodiments, the sample comprises any tissue or fragment thereof, excluding a thyroid tissue.

As used herein, the term “thyroid tissue” refers to any type of cell, tissue, fragments thereof, or any combination thereof, which at least partially make up the thyroid gland.

A person of ordinary skill in the art, would recognize types of cells, tissues, etc. making up the thyroid gland, and methods for identifying same.

In some embodiments, the extra thyroidal tissue or fragment thereof is selected form: a lymph node, a lung metastasis, a liver metastasis, a bone metastasis, a central nerve system (CNS) metastasis, or any combination thereof.

In some embodiments, a lymph node comprises a cervical lymph node, a mediastinal lymph node, an axillary lymph node, or a combination thereof.

In some embodiments, the lymph node is abnormally enlarged, abnormally structured, or both, compared to a control lymph node.

Methods for identifying and/or classifying a lymph node as being abnormally enlarged, abnormally structured, or both, are common and would be apparent to one of ordinary skill in the art.

As used herein, “abnormally enlarged” is having a volume increased by: at least 5%, at least 15%, at least 35%, at least 50%, at least 75%, at least 100%, at least 250%, at least 500%, at least 750%, or at least 1000%, compared to a control, or any value and range therebetween. Each possibility represents a separate embodiment of the invention.

In some embodiments, a control lymph node is derived or obtained from a healthy subject. In some embodiments, a control lymph node is derived or obtained from a subject comprising at least one healthy lymph node. In some embodiments, a control lymph node is a healthy (e.g., not abnormally enlarged, not abnormally structured, or both) lymph node derived or obtained from the same subject. In some embodiments, a control lymph node is derived or obtained from a subject not afflicted by a thyroid medical condition. In some embodiments, a control lymph node is derived or obtained from a subject afflicted with any medical condition excluding metastatic DTC, MTC, or both.

In some embodiments, detecting comprises qualitatively determining. In some embodiments, determining comprises quantitatively determining. In some embodiments, detecting comprises qualitatively and quantitatively determining.

In some embodiments, the method further comprises determining a progression stage of metastatic DTC in a subject. In some embodiments, the amount or level of a biomarker, e.g., thyroglobulin, that is related to a thyroid medical condition, e.g., DTC or metastatic DTC, correlates with the pathological state of the subject. In some embodiments, the amount or level of a biomarker, e.g., thyroglobulin, that is related to a thyroid medical condition, e.g., DTC or metastatic DTC, correlates with the progression stage of pathological state of DTC or metastatic DTC in the subject.

In some embodiments, the method further comprises a step of treating a subject diagnosed with metastatic DTC with an effective amount of anti-metastatic DTC therapy. In some embodiments, the herein disclosed method is for diagnosing and treating metastatic DTC in a subject in need thereof.

As used herein, the term “anti-metastatic DTC therapy” encompasses any conventional medicine means that is suitable for the treatment and/or alleviation of at least one symptom associated with metastatic DTC.

Compounds and methods for treating or alleviating symptoms associated with metastatic DTC, are common and would be apparent to one of ordinary skill in the art. Non-limiting examples for such therapy includes, but are not limited to, surgery, chemotherapy, radiology, and others.

In some embodiments, treating comprises surgically removing an enlarged cervical lymph node of the subject.

In some embodiments, treating comprises surgically removing at least a portion of a thyroid of the subject.

In some embodiments, treating comprises surgically removing a metastasis from the subject. In some embodiments, a metastasis is removed from a site selected from: lung, liver, bone, central nerve system (CNS), or any combination thereof.

In some embodiments, treating comprises administering to the subject a therapeutically effective amount of a drug suitable for DTC therapy.

In some embodiments, treating comprises subjecting the subject to a therapeutically effective amount of radiotherapy.

In some embodiments, treating comprises any combination of: surgically removing an enlarged cervical lymph node of a subject, surgically removing at least a portion of a thyroid of a subject, surgically removing a metastasis from a site selected from: lung, liver, bone, CNS, or any combination thereof, administering to a subject a therapeutically effective amount of a drug suitable for DTC therapy, and subjecting a subject to a therapeutically effective amount of radiotherapy.

In some embodiments, a drug suitable for DTC therapy drug is selected from: Vandetanib, Cabozantinib-S-Malate, Dabrafenib Mesylate, Doxorubicin Hydrochloride, Lenvatinib Mesylate, Trametinib, Sorafenib Tosylate, Selpercatinib, or any combination thereof.

In some embodiments, radiotherapy comprises internal radiotherapy, external radiotherapy, or a combination thereof.

In some embodiments, internal radiotherapy comprises radiolabeled iodine.

### Method of Diagnosing MTC

According to some embodiments, there is provided a method for diagnosing medullary thyroid carcinoma (MTC) in a subject.

In some embodiments, the method comprises the steps of: (a) providing a sample comprising a thyroidal tissue or a fragment thereof derived from the subject; and loading the sample from step (a) to the herein disclosed, and detecting a signal produced by the substrate molecule.

In some embodiments, the detection of a signal is indicative of a presence of a biomarker of MTC in the sample. In some embodiments, the presence of the biomarker of MTC in the sample is indicative of a cancerous thyroidal parafollicular cell being present in the sample, thereby diagnosing MTC in the subject.

In some embodiments, the lack of a detectable signal (e.g., negative detection, no detection, or any equivalent thereof) is indicative of an absence of a biomarker of MTC in the sample. In some embodiments, the absence of the biomarker of MTC in the sample is indicative of the absence of a cancerous thyroidal parafollicular cell in the sample or that a cancerous thyroidal parafollicular cell is absent from the sample, thereby determining the subject is not afflicted with MTC or the subject is MTC-free.

In some embodiments, the method is directed to diagnosing MTC in a sample derived or obtained from the subject. In some embodiments, the method is directed to in vitro or ex vivo diagnosing of MTC.

In some embodiments, in vitro or ex vivo is in a tube or a plate comprising a biological sample obtained or derived from a subject. In some embodiments, a biological sample comprises a cell, a tissue, an organ, a biopsy, an extract thereof, a portion thereof, a homogenate thereof, a fraction thereof, or any combination thereof.

In some embodiments, a thyroidal tissue or fragment thereof comprises a lymph node.

In some embodiments, the method further comprises determining a progression stage of MTC in a subject. In some embodiments, the amount or level of a biomarker, e.g., calcitonin, that is related to a thyroid medical condition, e.g., MTC, correlates with the pathological state of the subject. In some embodiments, the amount or level of a biomarker, e.g., calcitonin, that is related to a thyroid medical condition, e.g., MTC, correlates with the progression stage of pathological state of MTC in the subject.

In some embodiments, the method further comprises a step of treating a subject diagnosed with MTC with an effective amount of anti MTC therapy. In some embodiments, the herein disclosed method is for diagnosing and treating MTC in a subject in need thereof.

As used herein, the term “anti MTC therapy” encompasses any conventional medicine means that is suitable for the treatment and/or alleviation of at least one symptom associated with MTC.

Compounds and methods for treating or alleviating symptoms associated with MTC, are common and would be apparent to one of ordinary skill in the art. Non-limiting examples for such therapy includes, but are not limited to, surgery, chemotherapy, radiology, and others.

In some embodiments, treating comprises: surgically removing an enlarged cervical lymph node of a subject, surgically removing at least a portion of a thyroid of a subject, surgically removing a metastasis from a site selected from: lung, liver, bone, CNS, or any combination thereof, administering to a subject a therapeutically effective amount of a drug suitable for MTC therapy, subjecting a subject to a therapeutically effective amount of external radiotherapy, or any combination thereof.

In some embodiments, treating comprises surgically removing the thyroid of a subject. In some embodiments, treating comprises surgically removing the entire thyroid of a subject. In some embodiments, treating comprises the complete removal of a thyroid of a subject by means of a surgery or an operation.

In some embodiments, a drug suitable for MTC therapy is selected from: Vandetanib, Cabozantinib-S-Malate, Dabrafenib Mesylate, Doxorubicin Hydrochloride, Lenvatinib Mesylate, Trametinib, Sorafenib Tosylate, Selpercatinib, or any combination thereof.

As used herein, “removing at least a portion of a thyroid” comprises removing any portion of 1% to 99% of the thyroid gland, by weight or by volume.

### A Method for Determining Presence of a Biomarker of a Thyroid Medical Condition

According to some embodiments, there is provided a method for determining the presence of a biomarker of a thyroid medical condition in a biological sample, comprising the steps: (a) contacting the herein disclosed device with a biological sample; and (b) detecting the presence of a signal produced by a substrate molecule, wherein detection of a signal is indicative of a presence of a biomarker in the biological sample, thereby determining the presence of a biomarker of a thyroid medical condition in the biological sample.

As used herein, the terms “contacting” and “loading” are interchangeable, and refer to the application of a sample to a designated surface on the device of the invention, as would be apparent to one of ordinary skill in the art.

In some embodiments, a thyroid medical condition comprises thyroid cancer, a metastasis thereof, or a combination thereof.

As used herein, the term “biological sample” refers to a physical specimen obtained or derived from any animal. In another embodiment, biological sample is obtained from a mammal. In another embodiment, biological sample is obtained from a human. In another embodiment, biological sample is obtained well within the capabilities of those skilled in the art. The biological sample includes, but not limited to, biological fluids such as serum, plasma, vitreous fluid, lymph fluid, synovial fluid, follicular fluid, seminal fluid, amniotic fluid, milk, whole blood, urine, cerebrospinal fluid, saliva, sputum, tears, perspiration, mucus, and tissue culture media, including tissue extracts such as homogenized tissue, and cellular extracts. In another embodiment, a biological sample is a biopsy. In another embodiment, a biological sample is a resected tumor. In another embodiment, a biological sample includes histological sections processed as known by one skilled in the art. The terms sample and biological sample are used herein interchangeably.

### A Kit

According to some embodiments, there is provided a kit for diagnosing a thyroid medical condition comprising at least 4 sections, comprising: (a) a section 1, a section 2, a section 3, and a section 4; (b) at least one biomarker of a thyroid medical condition selected from: thyroglobulin, calcitonin, a para-thyroid hormone, fragments thereof, and any combination thereof; (c) at least one probing molecule linked to a reporter molecule and having specific binding affinity to the at least one biomarker or a fragment thereof, wherein the reporter molecule generates: chemically-, electrically-, or physically-detectable reaction; and (d) a substrate molecule reacting in the presence of the reporter molecule.

In some embodiments, the kit further comprises a calibration area.

In some embodiments, the kit further comprises instructions for depositing: (a) section 2 with the reporter molecule; (b) section 3 with the at least one biomarker of a thyroid medical condition; and (c) section 4 with the substrate molecule.

In some embodiments, the at least one biomarker comprises a peptide comprising an amino acid sequence set forth in SEQ ID NO: 1, SEQ ID NO:2, or a fragment thereof.

In some embodiments, the thyroid medical condition comprises thyroid cancer.

In some embodiments, thyroid cancer comprises metastatic DTC, MTC, or both.

In some embodiments, the at least one probing molecule is selected from any one of: (a) 138596-AF or SC-366977; and (b) DCABH-5057, MBS2107026, MBS2042771, MBS6250357, or MBS6250358.

In some embodiments, the at least one probing molecule is NB 110-8083.

### General

As used herein the term “about” refers to +10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments unless the embodiment is inoperative without those elements.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

The following studies were approved by a Helsinki committee (approval number 0170-17-SOR). Subjects were offered to participate and to sign the informed consent form when a relevant clinical question was asked (e.g., Is this suspicious cervical lymph node a metastasis of DTC origin?). During the study, the inventors performed both, the routine evaluation as recommended by the guidelines (GL), and in addition tested there herein disclosed thyroid medical condition point-of-care (POC) assay. All clinical decisions were made according to the current GL, and the results of the novel POC assay were interpreted retrospectively.

### Example 1

### Case Study 1

A 53-year-old woman was referred to the endocrine unit at the Soroka university medical center (Beer-Sheva, Israel) to perform FNAB from a highly suspicious 1.5 cm thyroid nodule. During ultrasound (US) evaluation prior to the procedure, a large and cystic right cervical lymph node was found, approximately 3 cm in its largest diameter. Initially, a FNAWF-Tg and FNAB from the suspicious lymph node were performed. Thereafter, the inventors completed a FNAB from the thyroid nodule.

The result of the FNAWF-Tg using the herein disclosed POC assay was positive within 2 minutes. The formal immunoassay according to the state of the art analysis, provided two days later, corroborated with the POC result of Tg levels of >30,000 ng/mL. Three weeks later, the inventors received the cytological results, concluding—atypia of undetermined significance (Bethesda 3) from the thyroid nodule and papillary structures typical of PTC from the metastatic lymph node.

Accordingly, the subject was referred to complete thyroidectomy with bilateral central and right lateral compartments neck dissection. The final pathological results confirmed the diagnosis, being a metastatic PTC (T1N1BMx) stage 3 disease.

### Example 2

### Case Study 2

A 40-year-old woman followed in the endocrine unit endocrine unit at the Soroka university medical center (Beer-Sheva, Israel) for Grave's disease, was referred to FNAB from a highly suspicious, 1.7 cm, left thyroid nodule. During US neck evaluation prior to the procedure, an oval, hypoechoic, 1.2 cm, left level 3, lymph node, was observed, moderately suspicious as a metastatic lymph node. A FNAWF-Tg from this lymph node was performed as well as FNAB from the suspicious left thyroid nodule.

The result of the FNAWF-Tg was negative using the herein disclosed POC assay. The formal immunoassay confirmed the result, according to which, Tg was undetectable. The cytological diagnosis from the left thyroid nodule was atypia of undetermined significance (Bethesda 3). Following a discussion with the subject's endocrinologist, the subject was referred to left thyroid lobectomy without neck dissection. The final pathological result was 1.5 cm PTC with low risk features, T1BN0BMx, stage 1 disease.

### Example 3

### Case Study 3

A 71-year-old woman was referred to the endocrine unit endocrine unit at the Soroka university medical center (Beer-Sheva, Israel) for the evaluation of a suspected recurrent PTC. Total thyroidectomy was performed 10 years ago for PTC followed by iodine ablation. Six months prior to the current visit, serum Tg levels became detectable and rose rapidly. Neck US examination revealed left neck pathological lymphadenopathy at levels 3, 4, and 6. A FNAWF-Tg and FNAB from the largest lymph node were performed.

The result of the FNAWF-Tg was positive using the herein disclosed POC assay. The formal immunoassay according to the state-of-the-art analysis, provided two days later, corroborated with the POC result of Tg levels of 83 ng/ml.

Three weeks later, the cytological results confirmed the diagnosis of metastatic PTC. Three weeks later, the inventors received the cytological results, concluding—metastatic PTC.

The subject was referred to bilateral central and left lateral compartments neck dissection. The final pathological results showed that out of 31 lymph nodes, 19 were metastatic, some of which with extra-nodal extension.

### Example 4

### Case Study 4

A 58-year-old man was scheduled for a third surgery for recurrent PTC. He was previously operated twice and received 250 millicurie of radioactive iodine in 2 fractions. Recent US revealed few pathologic lymph nodes in his left central and lateral cervical compartments. The largest lymph node, about 2 cm in diameter, located to the lower pole of left level 6, was determined as metastatic PTC, using FNAWF-Tg and FNAB. During the operation, dissection of the central compartment was performed first. Moving to the left lateral compartment it was unclear if few enlarged lymph nodes represented metastatic or reactive lymph nodes, and therefore, a representative lymph node was sample for subsequent analysis (e.g., as a frozen section). Simultaneously, FNAWF-Tg using the herein disclosed POC assay was performed, which provided a positive result within as little as 2 minutes. According to the study protocol, the inventors waited 40 minutes until the formal frozen section result was ready which confirmed that the lymph node was involved by PTC. Hence, formal left lateral compartment dissection was executed. Relying on the herein disclosed POC FNAWF-Tg could have saved 40 minutes of operating room time, staff resources, and materials, e.g., anesthetics, etc.

### Example 5

### Case Study 5

A 40-year-old woman was evaluated in the endocrine unit endocrine unit at the Soroka university medical center (Beer-Sheva, Israel) for symptomatic primary hyperparathyroidism. US and nuclear imaging failed to localize a parathyroid adenoma (PTA). However, a 2 cm, right thyroid nodule with benign features was identified using US. Since technetium-99m methoxyisobutylisonitrile (99mTc-MIBI) uptake by this nodule was high, the possibility of intrathyroidal PTA was considered. Following FNAB of the suspected thyroid nodule, FNAWF-Tg and washout for parathyroid hormone (PTH) were performed.

The result of the FNAWF-Tg using the herein disclosed POC assay was negative. The formal immunoassay according to the state of the art analysis confirmed that Tg was undetectable. The level of PTH in the washout fluids exceeded 6,000 pg/mL. The cytological result was “non-diagnostic”. However, the final pathological result confirmed the diagnosis of intrathyroidal PTA.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Following the above demonstrations, the inventors have concluded the following significant points: (1) POC assay for Tg can allow diagnosis of metastatic DTC at the same visit for the FNAB procedure, thus may shorten the time course needed for definite treatment and eliminate the period of uncertainty for the patient. This may also increase the yield of FNAB; (2) A positive result for Tg from a suspicious cervical lymph node may impact treatment decisions in a way that abolish the need for additional biopsies from thyroid nodules, since this is enough as an indication for total thyroidectomy and neck dissection; (3) A positive result from a suspicious cervical lymph node found during surgery, may allow the surgeon to shorten the surgery, e.g., by 30-45 minutes, which corresponds to the time needed to get a result from a frozen section; and (4) In cases of primary hyperparathyroidism (PHPT) without clear localization following US and nuclear medicine imaging, when the diagnosis of intrathyroidal PTA is considered, a negative result for Tg using the herein disclosed Tg POC assay from the nodule in question, increases the probability that this nodule represents intrathyroidal PTA. This information added to the FNAB for cytology, may assist the cytologist to establish the correct diagnosis of a PTA.

### Example 6

### Probing Molecule Suitability

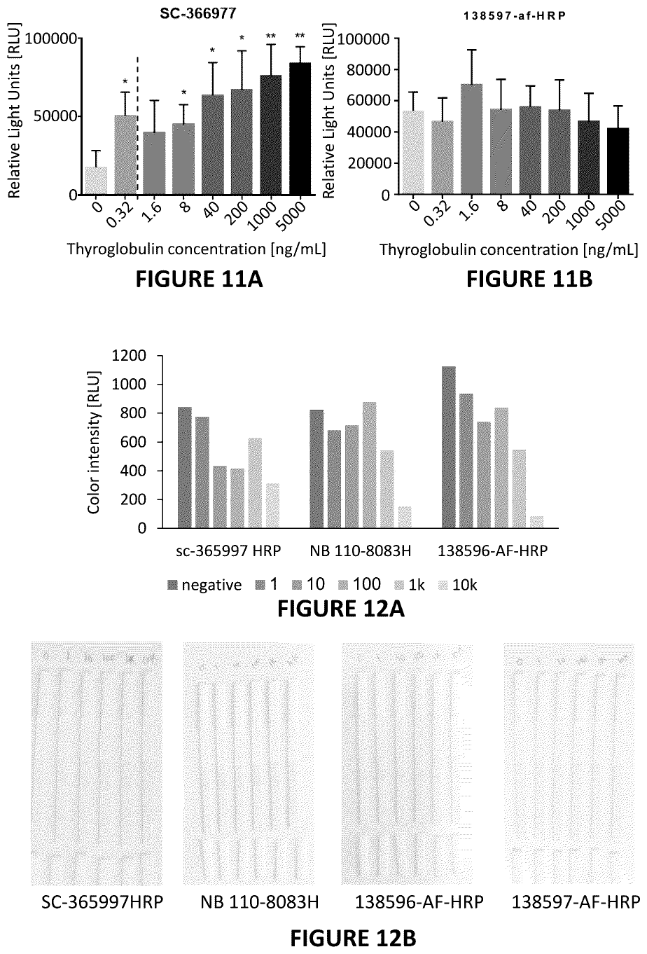

The inventors have compared the suitability of several antibodies capable of targeting thyroglobulin to be used in the herein disclosed device and respective methods. Indeed, the inventors have shown some anti-thyroglobulin antibodies were superior to others under the conditions and set-up disclosed herein (FIG. 11). For example, when using SC-366977 as a probing molecule, the signal generated in the assay had increased in a dose dependent manner of thyroglobulin concentration.

Further, after compiling 4 mm width strips composed of GFB-R4 sample pad, 1 cm PT-R5 conjugate pad, 4 cm CN95 nitrocellulose membrane and 4.1 cm AP 080 absorbent pad, a 1 μl dot of thyroglobulin 0.5 mg/mL in PBS was dried onto the nitrocellulose for 10 minutes at 50° C. Each strip was dipped in a well containing 80 μl of 0.04 μg/ml anti-thyroglobulin-HRP of different types, along with thyroglobulin in a concentration ranging from 0 to 10,000 ng/ml, in a PBS-T 0.05% w/v solution. Twenty (20) minutes after, TMB was applied to each strip to reveal the location of HRP.

Among the different antibodies used, 138596-AF-HRP showed great sensitivity in the competitive assay. Accordingly, 138596-AF-HRP is suggested as a promising antibody for the capture flow assay and device disclosed herein.

### Example 7

### Signal Lines Development

The inventors have further optimized signal development. In traditional assays, the antibodies end up attached to a solid phase, while an excess of substrate solution is applied over a large area. The substrate then reacts with the reporter to signal the location and quantity of the antibodies. However, in the developed assay, the platform is designed in such a way that the substrate awaits the antibodies to react in its position while the antibodies migrate through. Due to the nature of the enzyme-substrate reaction, each antibody-HRP can generate a signal at more than one line of the substrate, because the reaction does not attach the substrate to the HRP but activates it in the location of the reaction.

Three different chromogens were screen, to be used as a colorimetric substrate that react with the HRP reporter-conjugated to the anti-hTgs, while it migrates through the herein disclosed device. After drying the substrate, the antibody-HRP solution with H₂O₂ was applied to each nitrocellulose membrane dried with chromogen. DAB was found to present the strongest reaction to the presence of HRP after the drying process.

To further explore the option of signal amplification, imidazole in a range of concentrations was added to the running buffer. The inventors found that an increase in color intensity over the nitrocellulose membrane as the concentration of imidazole was increased from 0 to 28 mg/mL. However, at higher concentrations, the color signal declines up until the highest concentration tested of 70 mg/mL.

The inventors further attempted to restrict the color signal reaction migration.

The inventors showed that the reaction of the substrate line on the nitrocellulose, becomes more defined and less smeared with a concurrent increase in signal intensity upon the addition of PVA to the substrate solution. The signal intensity reaches a maximum at 0.25% w/v PVA and then is seen to decrease when more PVA is added to the solution.

To further control the localization of the color product on the nitrocellulose, the inventors have examined PEG.

PEG 8K and PEG 20K were found to restrict the migration of the substrate product over the nitrocellulose membrane. A dose-dependent effect was observed, where the higher concentration of PEG resulted in a more discrete signal. As the signal contracts, it also exhibits a more concentrated and stronger intensity of the signal.

### Example 8

### Thyroglobulin Point of Care Assay for Rapid Detection of Metastatic Differentiated Thyroid Carcinoma—A Pilot Study

The inventors assessed the diagnostic accuracy of the herein disclosed device, and methods of using same, as a qualitative point-of-care assay for thyroglobulin (POC-Tg). Specifically, the inventors examined whether the device is able to detect Tg in a needle washout of a suspicious lymph node (LN) within 10 minutes.

The inventors have set the limit of detection for Tg: Equal to 5 ng/ml following the accepted dilution.

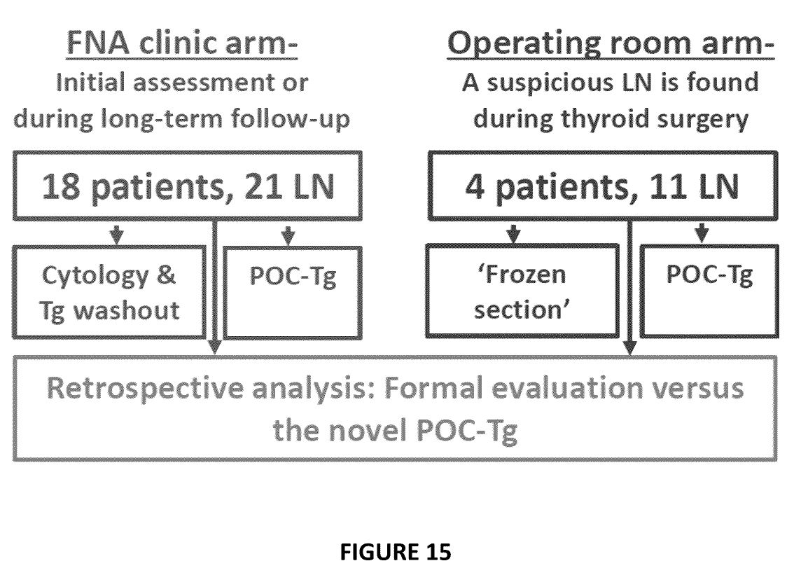

Two clinical settings were performed, as described in FIG. 15.

The inventors have successfully tested 22 suspected lymph nodes in the study. The results showed 100% compatibility to the standard immunoassay and 95% compatibility to cytology (one sample with low levels of Tg under 5 ng/ml was found positive in final pathology). The results are summarized herein below in Table 1.

TABLE 1
Results summary

| | Final diagnosis | | |
|---|---|---|---|
| The POC-Tg | DTC Metastasis | Benign LN | Total |
| Positive | 10 | 0 | 10 |
| Negative | 1* | 10 | 11 |
| Total | 11 | 10 | 21 |
| | Sensitivity 91% | Specificity 100% | |

Positive predictive value (PPV) - 100%, negative predictive value (NPV) - 91%, diagnostic accuracy - 95%; (*POC-Tg-Negative; Formal Tg washout - 1.33 ng/mL; Cytology- PTC MTS.)

The inventors have also tested 11 samples in the operation room (4 positive and 7 negative samples) with 100% compatibility to pathology (‘frozen sections’).

All publications, patents and patent applications mentioned in this specification are herein incorporated in their entirety by reference into the specification, to the same extent as if each individual publication, patent or patent application was specifically and individually indicated to be incorporated herein by reference. In addition, citation, or identification of any reference in this application shall not be construed as an admission that such reference is available as prior art to the present invention. To the extent that section headings are used, they should not be construed as necessarily limiting.

1. A device for rapid diagnosis of a thyroid medical condition comprising at least 4 sections comprising: section 1, section 2, section 3, and section 4, sequentially and linearly coupled to each other, wherein:
a. section 1 comprises a sample collecting surface;
b. section 2 comprises at least one probing molecule having specific binding affinity to at least one biomarker of a thyroid medical condition being selected from the group consisting of: calcitonin, a parathyroid hormone, fragments thereof, and any combination thereof, wherein said at least one probing molecule is linked to a reporter molecule capable of generating a trigger;
c. section 3 comprises a surface functionalized with said at least one biomarker of a thyroid medical condition; and
d. section 4 comprises a surface comprising a substrate molecule capable of producing a signal upon contacting with said at least one reporter molecule linked to said probing molecule,
wherein sections 1 to 4 are in liquid communication allowing flow of liquid sequentially from said sections 1 to 4.

2. A device for rapid diagnosis of a thyroid medical condition comprising at least 4 sections comprising: section 1, section 2, section 3, and section 4, sequentially and linearly coupled to each other, wherein:
a. section 1 comprises a sample collecting surface;
b. section 2 comprises at least one probing molecule having specific binding affinity to at least one biomarker of a thyroid medical condition being selected from the group consisting of: thyroglobulin, fragments thereof, and any combination thereof, wherein said at least one probing molecule is linked to a reporter molecule capable of generating a trigger, and wherein said at least one probing molecule comprises an antibody selected from the group consisting of: 138596-AF, and SC-366977;
c. section 3 comprises a surface functionalized with thyroglobulin; and
d. section 4 comprises a surface comprising a substrate molecule capable of producing a signal upon contacting with said at least one reporter molecule linked to said probing molecule,
wherein sections 1 to 4 are in liquid communication allowing flow of liquid sequentially from said sections 1 to 4.

3. The device of claim 1, wherein said sections 1 to 4 are arranged along a horizontal axis, and optionally wherein said flow is a lateral flow.

4. (canceled)

5. The device of claim 1, wherein said sections 1 to 4 are arranged along a vertical axis, and optionally wherein said flow is a longitudinal flow.

6. (canceled)

7. The device of claim 1, wherein said thyroid medical condition comprises thyroid cancer, metastases thereof, or a combination thereof.

8. The device of claim 1, wherein said at least one biomarker is a biomarker of medullary thyroid carcinoma (MTC), and optionally wherein said at least one biomarker of MTC is a peptide comprising an amino acid sequence set forth in SEQ ID NO: 1, or a fragment thereof.

9. (canceled)

10. The device of claim 1, wherein said at least one probing molecule comprises an antibody selected from the group consisting of: DCABH-5057, MBS2107026, MBS2042771, MBS6250357, and MBS6250358.

11. The device of claim 2, wherein said at least one biomarker is a biomarker of differentiated thyroid carcinoma (DTC), and optionally wherein said at least one biomarker of DTC is a peptide comprising an amino acid sequence set forth in SEQ ID NO: 2, or a fragment thereof.

12. (canceled)

13. The device of claim 1, wherein said reporter molecule is selected from the group consisting of: an enzyme, a radioactive molecule, a luminescent compound, a fluorescent compound, a magnetic particle, an electro-chemiluminescent compound, a fluorescence transducing aptamer and an electrochemically active compound.

14. The device of claim 1, further comprising any one of: (i) a calibration area disposed between said section 2 and said section 3, wherein said calibration area comprises a surface in contact with said substrate molecule; and (ii) a detection unit in operable communication with said device, and wherein said detection unit is configured to detect said signal, and optionally wherein said detection unit comprises an element selected form the group consisting of: an active-pixel sensor (APS), an electrode, an excitation source with active-pixel sensor, and any combination thereof.

15.-16. (canceled)

17. The device of claim 1, wherein said rapid diagnosis of said thyroid medical condition is provided within 1 minute to 30 minutes.

18. A method for diagnosing metastatic differentiating thyroid carcinoma (DTC) in a subject, comprising the steps of:
a. providing a sample comprising an extra thyroidal tissue or a fragment thereof derived from said subject; and
b. loading said sample from step (a) to the device of claim 2, and detecting a signal produced by said substrate molecule, wherein detection of said signal is indicative of a presence of a biomarker of DTC in said sample, and wherein the presence of said biomarker of DTC in said sample is indicative of a cancerous thyroidal cell being present in said sample,
thereby diagnosing metastatic DTC in said subject.

19. The method of claim 18, wherein said DTC comprises any one of: papillary thyroid carcinoma (PTC), follicular thyroid carcinoma (FTC), and a combination thereof, and optionally wherein any one of: (i) said sample is devoid of a thyroid tissue; (ii) said extra thyroidal tissue or fragment thereof is selected form the group consisting of: a lymph node, a lung metastasis, a liver metastasis, a bone metastasis, a central nerve system (CNS) metastasis, and any combination thereof; (iii) said lymph node is a cervical lymph node, a mediastinal lymph node, or an axillary lymph node; (iv) said lymph node is abnormally enlarged, abnormally structured, or both, compared to a control lymph node; (v) said detecting comprises qualitatively determining, quantitatively determining, or both; and (vi) any combination of (i) to (v).

20.-24. (canceled)

25. The method of claim 18, further comprising determining a progression stage of said metastatic DTC in said subject.

26. The method of claim 18, further comprising a step of treating said subject diagnosed with metastatic DTC with an effective amount of anti-metastatic DTC therapy, and optionally wherein any one of: (i) said anti-metastatic DTC therapy comprises: surgically removing an enlarged cervical lymph node of said subject, surgically removing at least a portion of a thyroid of said subject, surgically removing a metastasis from a site selected from the group consisting of: lung, liver, bone, CNS, and any combination thereof, administering to said subject a therapeutically effective amount of a drug suitable for DTC therapy, subjecting said subject to a therapeutically effective amount of radiotherapy, or any combination thereof; (ii) said drug is selected from the group consisting of: Vandetanib, Cabozantinib-S-Malate, Dabrafenib Mesylate, Doxorubicin Hydrochloride, Lenvatinib Mesylate, Trametinib, Sorafenib Tosylate, and Selpercatinib; (iii) said radiotherapy comprises internal radiotherapy or external radiotherapy; (iv) said internal radiotherapy comprises radiolabelled iodine; and (v) any combination of (i) to (iv).

27.-30. (canceled)

31. A method for diagnosing medullary thyroid carcinoma (MTC) in a subject, comprising the steps of:
a. providing a sample comprising a thyroidal tissue or a fragment thereof derived from said subject; and
b. loading said sample from step (a) to the device of claim 1, and detecting a signal produced by said substrate molecule, wherein detection of said signal is indicative of a presence of a biomarker of MTC in said sample, and wherein the presence of said biomarker of MTC in said sample is indicative of a cancerous thyroidal parafollicular cell being present in said sample,
thereby diagnosing metastatic MTC in said subject.

32. The method of claim 31, wherein said MTC comprises metastatic MTC, and optionally wherein any one of: (i) said thyroidal tissue or fragment thereof comprises a lymph node; (ii) said lymph node is a cervical lymph node, a mediastinal lymph node, or an axillary lymph node; (iii) said lymph node is abnormally enlarged, abnormally structured, or both, compared to a control lymph node; and (iv) any combination of (i) to (iii).

33.-35. (canceled)

36. The method of claim 31, wherein said detecting comprises qualitatively determining, quantitatively determining, or both, and optionally wherein said method further comprises determining a progression stage of said MTC in said subject.

37. (canceled)

38. The method of claim 31, further comprising a step of treating said subject diagnosed with MTC with an effective amount of anti MTC therapy, and optionally wherein any one of: (i) said treating comprises: surgically removing an enlarged cervical lymph node of said subject, surgically removing at least a portion of a thyroid of said subject, surgically removing a metastasis from a site selected from the group consisting of: lung, liver, bone, CNS, and any combination thereof, administering to said subject a therapeutically effective amount of a drug suitable for MTC therapy, subjecting said subject to a therapeutically effective amount of an external radiotherapy, or any combination thereof; (ii) said drug is selected from the group consisting of: Vandetanib, Cabozantinib-S-Malate, Dabrafenib Mesylate, Doxorubicin Hydrochloride, Lenvatinib Mesylate, Trametinib, Sorafenib Tosylate, and Selpercatinib; and (iii) both (i) and (ii).

39.-40. (canceled)

41. A kit for diagnosing a thyroid medical condition comprising at least 4 sections, comprising:
a. a section 1, a section 2, a section 3, and a section 4;
b. at least one biomarker of a thyroid medical condition selected from the group consisting of: thyroglobulin, calcitonin, a para-thyroid hormone, fragments thereof, and any combination thereof;
c. at least one probing molecule linked to a reporter molecule and having specific binding affinity to said at least one biomarker or a fragment thereof, wherein said reporter molecule generates: chemically-, electrically-, or physically-detectable reaction; and
d. a substrate molecule reacting in the presence of said reporter molecule,
and optionally wherein:
i. said kit further comprises a calibration area;
ii. said kit further comprises instructions for depositing: said section 2 with said reporter molecule, said section 3 with said at least one biomarker of a thyroid condition, and said section 4 with said substrate molecule;
iii. said at least one biomarker comprises a peptide comprising an amino acid sequence set forth in SEQ ID NO: 1, SEQ ID NO:2, or a fragment thereof;
iv. said thyroid medical condition comprises thyroid cancer;
v. said thyroid cancer comprises metastatic DTC, MTC, or both; or
vi. said at least one probing molecule is selected from any one of: 138596-AF or SC-366977; and DCABH-5057, MBS2107026, MBS2042771, MBS6250357, or MBS6250358.

42.-47. (canceled)
